(19) United States
(12) Patent Application Publication
(10) Pub. No.: US 2009/0258858 A1
(43) Pub. Date: Oct. 15, 2009

(54) MUSCARINIC ACETYLCHOLINE RECEPTOR ANTAGONISTS

(76) Inventors: Jakob Busch-Petersen, King of Prussia, PA (US); Jeffrey Charles Boehm, King of Prussia, PA (US); Huijie Li, King of Prussia, PA (US); John J. Taggart, King of Prussai, PA (US); Hong Xing Yan, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/578,000

(22) PCT Filed: Oct. 28, 2005
(86) PCT No.: PCT/US05/39209

§ 371 Date: Apr. 26, 2007

Related U.S. Application Data
(60) Provisional application No. 60/623,558, filed on Oct. 29, 2004.

(51) Int. Cl. A61K 31/17; C07C 273/00; C07D 487/08; C07D 221/22; C07D 223/16; A61K 31/403; A61K 31/435; A61K 31/55; A61P 11/00

(52) U.S. Cl. 514/216; 564/47; 548/428; 546/112; 540/594; 540/582; 514/595; 514/411; 514/299; 514/217.01

(57) ABSTRACT

Muscarinic Acetylcholine receptor antagonists and methods of using them are provided.

### FIELD OF THE INVENTION

This invention relates to novel benzazepine compounds, pharmaceutical compositions, processes for their preparation, and use thereof in treating muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs, and these receptors can mediate both inhibitory and excitatory actions. For example, in smooth muscle found in the airways, bladder and gastrointestinal tract, M₃ mAChRs mediate contractile responses (1989. The Muscarinic Receptors. The Humana Press, Inc., Clifton, N.J.).

Muscarinic acetylcholine receptor dysfunction has been noted in a variety of different pathophysiological states. For instance, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation. This mAChR dysfunction results in airway hyperreactivity mediated by increased stimulation of M₃ mAChRs. Similarly, inflammation of the gastrointestinal tract in inflammatory bowel disease (IBD) results in M₃ mAChR-mediated hypermotility (Oprins, J. C. J., HP. Meijer, and J. A. Groot. 2000. Tumor Necrosis Factor-{alpha} Potentiates Ion Secretion Induced by Muscarinic Receptor Activation in the Human Intestinal Epithelial Cell Line HT29cl.19A. Ann NY Acad Sci 915:102-106). Incontinence due to bladder hypercontractility has also been demonstrated to be mediated through increased stimulation of M₃ mAChRs. Thus the identification of subtype-selective mAChR antagonists may be useful as therapeutics in these mAChR-mediated diseases.

Despite the large body of evidence supporting the use of anti-muscarinic receptor therapy for treatment of a variety of disease states, relatively few anti-muscarinic compounds are in use in the clinic. Thus, there remains a need for novel compounds that are capable of causing blockade at M₃ mAChRs. Conditions associated with an increase in stimulation of M₃ mAChRs, such as asthma, COPD, IBD and urinary incontinence would benefit by compounds that are inhibitors of mAChR binding.

### SUMMARY OF THE INVENTION

This invention provides for a method of treating a muscarinic acetylcholine receptor (mAChR) mediated disease, wherein acetylcholine binds to an mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent;

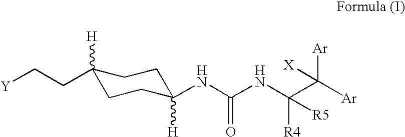

wherein:
cyclohexyl stereochemistry is cis or trans;
Y is an amine, or quaternary amine salt;
R⁴ and R⁵, are independently selected from the group consisting of a substituent selected from C₁₋₆ alkyl, aryl and arylC₁₋₆alkyl, C1-6 alkylaryl, heteroaryl, heteroalkyl, all optionally substituted, and H

### X is OH or CN

amine is selected from the group consisting of:

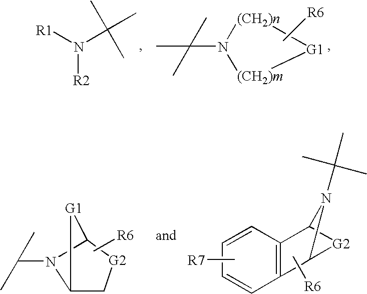

R1 and R2 are independently selected from a group consisting of H, alkyl, cycloalkyl, aryl, alkylaryl, alkylalkenyl, arylalkyl and arylalkenyl, all optionally substituted;
m is an integer from 0 to 6;
n is an integer from 0 to 5;
G1 and G2 are independently selected from, bond, (CH2)p, O, NR1, —CR1=CR1-, S(O)p, CO and CONR1-;
p is an integer from 0 to 2;
R6 and R7 are independently selected from ═R1, F, Cl, Br, CN, OR1, OCOR2, NR1R2 and NCOR2;
Ar is independently selected from the group consisting of aryl and heteroaryl, all optionally substituted;

Quaternary ammonium salt is selected from the group consisting of:

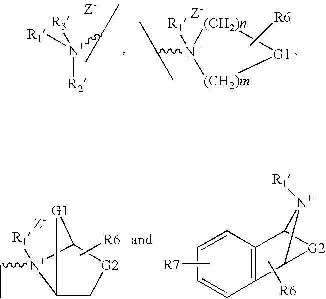

Z- is an anionic counterion including I⁻, Br⁻, HSO₄⁻ HCO_{2′}

R₁′, R₂′ and R₃′, are independently selected from a group consisting of H, alkyl, cycloalkyl, aryl, alkylaryl, alkylalkenyl, arylalkyl, arylalkenyl, all optionally substituted; and M, N, G1. G2 P, R6 and R7 are as defined herein above.

In the compounds of formula (I) above an alkyl group or moiety may be straight or branched. Alkyl groups which may be employed include methyl, ethyl, n-propyl, n-butyl, n-pentyl, n-hexyl and any branched isomers thereof such as isopropyl, t-butyl, sec-butyl, and the like.

R1, R2, R3, are independently selected from a group consisting of an aroyl, or aroylC₁₋₄alkyl, the aryl moiety may be selected from an optionally substituted phenyl ring or an optionally substituted 5- or 6-membered heterocyclic ring. In the groups R1, R2, R3 an aryl moiety may be optionally substituted by one or more substituents selected from hydrogen, halogen, amino, cyano, C₁₋₄alkyl, C₁₋₄alkylamino, C₁₋₄dialkylamino, C₁₋₄alkylamido, C₁₋₄alkanoyl, or R⁸R⁹NCO where each of R⁸ and R⁹ are, independently, selected from a group consisting of a hydrogen atom or C₁₋₄alkyl group.

An optionally substituted 5- or 6-membered heterocyclic aromatic ring, as defined for any of the groups Ar, or Ar′ may contain from 1 to 4 heteroatoms selected from O, N or S. When the ring contains 24 heteroatoms, one is preferably selected from O, N and S and the remaining heteroatoms are preferably N. Examples of 5 and 6-membered heterocyclic groups include furyl, thienyl, pyrrolyl, oxazolyl, thiazolyl, imidazolyl, oxadiazolyl, thiadiazolyl, pyridyl, triazolyl, triazinyl, pyridazyl, pyrimidinyl, pyrazolyl, isothiazolyl, and isoxazolyl.

Examples of bicyclic, for example bicyclic aromatic or heteroaromatic, ring systems for Ar include naphthyl, indazolyl, indolyl, benzofuranyl, benzothienyl, benzothiazolyl, benziridazolyl, benzoxazolyl, benzisoxazolyl, benzisothiazolyl, quinolinyl, quinoxolinyl, quinazolinyl, cinnolinyl, isoquinolinyl, pyrazolo[1,5-a]pyrimidyl; pyrrolo[3,2-b]pyridyl, pyrrolo[3,2-c]pyridyl, thieno[3,2-b]thiophenyl, 1,2-dihydro-2-oxo-quinolinyl, 3,4-dihydro-3-oxo-2H-benzoxazinyl, 1,2-dihydro-2-oxo-3H-indolyl.

The rings Ar or Ar′ may each independently be optionally substituted by one or more substituents selected from: a hydrogen or halogen atom, or a hydroxy, oxo, cyano, nitro, trifluoromethyl, C₁₋₄alkyl, C₁₋₄alkoxy, C₁₋₄alkylenedioxy, C₁₋₄alkanoyl, C₁₋₄alkylsulfonyl, C₁₋₄alkylsulfinyl, C₁₋₄alkylthio, R¹⁰SO₂N(R¹¹)—, R¹⁰R¹¹NSO₂—, R¹⁰R¹¹N—, R¹⁰O₂C—, R¹⁰R¹¹NC(O)—, or R¹⁰CON(R¹¹)— group wherein each of R¹⁰ and R¹¹ independently, selected from a group consisting of a hydrogen atom or a C₁₋₄ alkyl group, or R¹⁰R¹¹ together form a C₃₋₆ alkylene chain.

Alternatively, Ar and Ar′ may be optionally substituted by one or more 5- or 6-membered heterocyclic rings, as defined above, optionally substituted by a C₁₋₂ alkyl or R⁷R⁸N— group; wherein R⁷ and R⁸ are as defined above.

In the rings Ar and Ar′ substituents positioned ortho to one another may be linked to form a 5- or 6-membered ring.

It will be appreciated that for use in medicine the salts of formula (I) should be physiologically acceptable. Suitable physiologically acceptable salts will be apparent to those skilled in the art and include for example acid addition salts formed with inorganic acids eg. hydrochloric, hydrobromic, sulfuric, nitric or phosphoric acid; and organic acids eg. succinic, maleic, acetic, fumaric, citric, tartaric, benzoic, p-toluenesulfonic, methanesulfonic or naphthalenesulfonic acid. Other non-physiologically acceptable salts eg. oxalates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

The compounds of formula (I) can exist in the form of cis- and trans-isomers with respect to the configuration at the cyclohexyl ring. When A represents a group (c) the compounds may also exist as geometric isomers around the double bond. The present invention includes within its scope all such isomers, including mixtures. Preferably the compounds of the invention are in the trans configuration with respect to the cyclohexyl ring. For compounds of formula (I) where A represents a group (c), trans geometry of the double bond is preferred.

It is also preferred that the rings Ar are each independently optionally substituted by one or more substituents selected from: a hydrogen or halogen atom, cyano, methoxy, trifluoromethyl, methylenedioxy, acetyl, acetylamino, methylsulfonyl, methylsulfonyloxy, methylaminosulfonyl, methylsulfonylamino, or methylaminocarbonyl group.

Certain of the substituted heteroaromatic ring systems included in compounds of formula (I) may exist in one or more tautomeric forms. The present invention includes within its scope all such tautomeric forms, including mixtures.

The following terms, as used herein, refer to:
“halo”—all halogens, that is chloro, fluoro, bromo and iodo.
“Cl₁₋₁₀alkyl” or “alkyl”—both straight and branched chain moieties of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like.
“cycloalkyl” is used herein to mean cyclic moiety, preferably of 3 to 8 carbons, including but not limited to cyclopropyl, cyclopentyl, cyclohexyl, and the like.
“alkenyl” is used herein at all occurrences to mean straight or branched chain moiety of 2-10 carbon atoms, unless the chain length is limited thereto, including, but not limited to ethenyl, 1-propenyl, 2-propenyl, 2-methyl-1-propenyl, 1-butenyl, 2-butenyl and the like.
“aryl”—phenyl and naphthyl;
“heteroaryl” (on its own or in any combination, such as “heteroaryloxy”, or “heteroaryl alkyl”)—a 5-10 membered aromatic ring system in which one or more rings contain one or more heteroatoms selected from the group consisting of N, O or S, such as, but not limited, to pyrrole, pyrazole, furan, thiophene, quinoline, isoquinoline, quinazolinyl, pyridine, pyrimidine, oxazole, tetrazole, thiazole, thiadiazole, triazole, imidazole, or benzimidazole.
“heterocyclic” (on its own or in any combination, such as “heterocyclicalkyl”)—a saturated or partially unsaturated 4-10 membered ring system in which one or more rings contain one or more heteroatoms selected from the group consisting of N, O or S; such as, but not limited to, pyrrolidine, piperidine, piperazine, morpholine, tetrahydropyran, thiomorpholine, or imidazolidine. Furthermore, sulfur may be optionally oxidized to the sulfone or the sulfoxide.

“arylalkyl” or “heteroarylalkyl” or “heterocyclicalkyl” is used herein to mean C₁₋₁₀ alkyl, as defined above, attached to an aryl, heteroaryl or heterocyclic moiety, as also defined herein, unless otherwise indicated.

Particular compounds according to the invention include those specifically exemplified and named hereinafter,
N-(trans-4-{2-[(1R,8S)-11-azatricyclo[6.2.1.0˜2,7˜]undeca-2,4,6-trien-11-yl]ethyl}cyclohexyl)-N′-[(1S)-2-hydroxy-1-methyl-2,2-diphenylethyl]urea;
N-(trans-4-{2-[bis(phenylmethyl)amino]ethyl}cyclohexyl)-N′-[(1S)-2-hydroxy-1-methyl-2,2-diphenylethyl]urea;
N-(trans-4-{2-[bis(phenylmethyl)amino]ethyl}cyclohexyl)-N′-[(1R)-2-hydroxy-1-methyl-2,2-diphenylethyl]urea;
N-{trans-4-[2-(dicyclohexylamino)ethyl]cyclohexyl}-N′-[(1R)-2-hydroxy-1-methyl-2,2-diphenylethyl]urea;
N-{trans-4-[2-(dicyclohexylamino)ethyl]cyclohexyl}-N′-[(1S)-2-hydroxy-1-methyl-2,2-diphenylethyl]urea;
N-(trans-4-{2-[(1R,8S)-11-azatricyclo[6.2.1.0˜2,7˜]undeca-2,4,6-trien-11-yl]ethyl}cyclohexyl)-N′-[(1R)-2-hydroxy-1-methyl-2,2-diphenylethyl]urea;
N-(trans-4-{2-[(1R,5S)-3-azabicyclo[3.2.1]oct-3-yl]ethyl}cyclohexyl)-N′-[(1S)-2-hydroxy-1-methyl-2,2-diphenylethyl]urea;
N-(trans-4-{2-[(1R,4S)-2-azabicyclo[2.2.1]hept-2-yl]ethyl}cyclohexyl)-N′-[(1R)-2-hydroxy-1-methyl-2,2-diphenylethyl]urea;
N-(trans-4-{2-[(1R,5S)-8-azabicyclo[3.2.1]oct-8-yl]ethyl}cyclohexyl)-N′-[(1R)-2-hydroxy-1-methyl-2,2-diphenylethyl]urea;
N-{trans-4-[2-(1,3-dihydro-2H-isoindol-2-yl)ethyl]cyclohexyl}-N′-[(1R)-2-hydroxy-1-methyl-2,2-diphenylethyl]urea;
N-{trans-4-[2-(2,3-dihydro-1H-indol-1-yl)ethyl]cyclohexyl}-N′-[(1R)-2-hydroxy-1-methyl-2,2-diphenylethyl]urea;
N-(trans-4-{2-[(1s,4s)-7-azabicyclo[2.2.1]hept-7-yl]ethyl}cyclohexyl)-N′-[(1R)-2-hydroxy-1-methyl-2,2-diphenylethyl]urea;
N-[(1R)-2-hydroxy-1-methyl-2,2-diphenylethyl]-N′-(trans-4-{2-[7-(2-methylpropanoyl)-1,2,4,5-tetrahydro-3H-3-benzazepin-3-yl]ethyl}cyclohexyl)urea;
N-(trans-4-{2-[(1s,5s)-3-azabicyclo[3.2.2]non-3-yl]ethyl}cyclohexyl)-N′-[(1R)-2-hydroxy-1-methyl-2,2-diphenylethyl]urea;
N-(trans-4-{2-[(1R,5S)-3-azabicyclo[3.2.1]oct-3-yl]ethyl}cyclohexyl)-N′-[(1R)-2-hydroxy-1-methyl-2,2-diphenylethyl]urea;
N-(trans-4-{2-[(1R,5S)-3-hydroxy-8-azabicyclo[3.2.1]oct-8-yl]ethyl}cyclohexyl)-N′-[(1R)-2-hydroxy-1-methyl-2,2-diphenylethyl]urea;
Ethyl N-(2-{trans-4-[({[(1R)-2-hydroxy-1-methyl-2,2-diphenylethyl]amino}carbonyl)amino]cyclohexyl}ethyl)-N-methylglycinate;
N-{trans-4-[2-(3-azabicyclo[3.2.2]non-3-yl)ethyl]cyclohexyl}-N′-[(1S)-2-hydroxy-1-methyl-2,2-diphenylethyl]urea;
N-(trans-4-{2-[(1R,4S)-2-azabicyclo[2.2.1]hept-2-yl]ethyl}cyclohexyl)-N′-[(1S)-2-hydroxy-1-methyl-2,2-diphenylethyl]urea;
N-(trans-4-{2-[(1s,4s)-7-azabicyclo[2.2.1]hept-7-yl]ethyl}cyclohexyl)-N′-[(1S)-2-hydroxy-1-methyl-2,2-diphenylethyl]urea;
N-(trans-4-{2-[(1R,5S)-8-azabicyclo[3.2.1]oct-8-yl]ethyl}cyclohexyl)-N′-[(1S)-2-hydroxy-1-methyl-2,2-diphenylethyl]urea;
(1R,5S)-8-(2-{trans-4-[({[(1S)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
(1R,2S,4R,5S)-9-(2-{trans-4-[({[(1S)-2-hydroxy-1-methyl-2,2-diphenylethyl]amino}carbonyl)amino]cyclohexyl}ethyl)-3-oxa-9-azatricyclo[3.3.1.0˜2,4˜]non-7-yl 3-hydroxy-2-phenylpropanoate;
N-{trans-4-[2-(3,4-dihydro-1(2H)-quinolinyl)ethyl]cyclohexyl}-N′-[(1S)-2-hydroxy-1-methyl-2,2-diphenylethyl]urea;
N-{trans-4-[2-(3,4-dihydro-2(1H)-isoquinolinyl)ethyl]cyclohexyl}-N′-[(1S)-2-hydroxy-1-methyl-2,2-diphenylethyl]urea;
N-(trans-4-{2-[(1R,5S)-3-hydroxy-8-azabicyclo[3.2.1]oct-8-yl]ethyl}cyclohexyl)-N′-[(1S)-2-hydroxy-1-methyl-2,2-diphenylethyl]urea;
N-[(1S)-2-hydroxy-1-methyl-2,2-diphenylethyl]-N′-{trans-4-[2-(2-phenyl-1-pyrrolidinyl)ethyl]cyclohexyl}urea;
N-[(1S)-2-hydroxy-1-methyl-2,2-diphenylethyl]-N′-{trans-4-[2-(4-phenyl-1-piperidinyl)ethyl]cyclohexyl}urea;
N-(trans-4-{2-[(1R,8S)-4-azatricyclo[4.3.1.1˜3,8˜-]undec-4-yl]ethyl}cyclohexyl)-N′-[(1R)-2-hydroxy-1-methyl-2,2-diphenylethyl]urea;
1,1-dimethylethyl N-(2-{trans-4-[({[(1R)-2-hydroxy-1-methyl-2,2-diphenylethyl]amino}carbonyl)amino]cyclohexyl}ethyl)-N-methylglycinate;
N-[(1R)-2-hydroxy-1-methyl-2,2-diphenylethyl]-N′-{trans-4-[2-(2-phenyl-1-piperidinyl)ethyl]cyclohexyl}urea;
N-[(1R)-2-hydroxy-1 methyl-2,2-diphenylethyl]-N′-(trans-4-{2-[methyl(phenylmethyl)amino]ethyl}cyclohexyl)urea;
N-[(1R)-2-hydroxy-1-methyl-2,2-diphenylethyl]-N′-{trans-4-[2-(2-phenyl-1-pyrrolidinyl)ethyl]cyclohexyl}urea;
N-(2-{trans-4-[({[(1R)-2-hydroxy-1-methyl-2,2-diphenylethyl]amino}carbonyl)amino]cyclohexyl}ethyl)-N-methylglycine;
N-[(1R)-2-hydroxy-1-methyl-2,2-diphenylethyl]-N′-{trans-4-[2-(3-phenyl-1-pyrrolidinyl)ethyl]cyclohexyl}urea;
N-[(1R)-2-hydroxy-1-methyl-2,2-diphenylethyl]-N′-{trans-4-[2-(3-phenyl-1-piperidinyl)ethyl]cyclohexyl}urea;
9-(2-{trans-4-[({[(1R)-2-hydroxy-1-methyl-2,2-diphenylethyl]amino}carbonyl)amino]cyclohexyl}ethyl)-3-oxa-9-azatricyclo[3.3.1.0˜2,4˜]non-7-yl 3-hydroxy-2-phenylpropanoate;
N-[(1S)-2-hydroxy-1-methyl-2,2-diphenylethyl]-N′-(trans-4-{2-[(1R,5S)-3-hydroxy-3-phenyl-8-azabicyclo[3.2.1]oct-8-yl]ethyl}cyclohexyl)urea;
Ethyl N-(2-{trans-4-[({[(1S)-2-hydroxy-1-methyl-2,2-diphenylethyl]amino}carbonyl)amino]cyclohexyl}ethyl)-N-methylglycinate;
N-[(1S)-2-hydroxy-1-methyl-2,2-diphenylethyl]-N′-(trans-4-{2-[7-(2-methylpropanoyl)-1,2,4,5-tetrahydro-3H-3-benzazepin-3-yl]ethyl}cyclohexyl)urea;
N-[(1S)-2-hydroxy-1-methyl-2,2-diphenylethyl]-N′-{trans-4-[2-(3-phenyl-1-piperidinyl)ethyl]cyclohexyl}urea;
1-[(1S)-2-hydroxy-1-methyl-2,2-diphenylethyl]-3-{trans-4-[2-(3-phenyl-1-piperidinyl)ethyl]cyclohexyl}urea;
N-[(1S)-2-hydroxy-1-methyl-2,2-diphenylethyl]-N′-{trans-4-[2-(3-phenyl-1-pyrrolidinyl)ethyl]cyclohexyl}urea;
N-[(1S)-2-hydroxy-1-methyl-2,2-diphenylethyl]-N′-{trans-4-[2-(2-phenyl-1-piperidinyl)ethyl]cyclohexyl}urea;
(1R,8S)-11-(2-{trans-4-[({[(1R)-2-hydroxy-1-methyl-2,2-diphenylethyl]amino}carbonyl)amino]cyclohexyl}ethyl)-11-methyl-11-azoniatricyclo[6.2.1.0˜2,7˜]undeca-2,4,6-triene iodide;
(1R,8S)-11-(2-{trans-4-[({[(1S)-2-hydroxy-1-methyl-2,2-diphenylethyl]amino}carbonyl)amino]cyclohexyl}ethyl)-11-methyl-11-azoniatricyclo[6.2.1.0˜2,7˜]undeca-2,4,6-triene iodide;
1-(2-{trans-4-[({[(1R)-2-hydroxy-1-methyl-2,2-diphenylethyl]amino}carbonyl)amino]cyclohexyl}ethyl)-1-methyl-3-phenylpiperidinium iodide;
2-(2-{trans-4-[({[(1S)-2-hydroxy-1-methyl-2,2-diphenylethyl]amino}carbonyl)amino]cyclohexyl}ethyl)-2-methyl-2-azoniatricyclo[3.3.1.1˜3,7˜]decane iodide;
N-[(1R)-2-hydroxy-1-methyl-2,2-diphenylethyl]-N′-(trans-4-{2-[(1R,8S)-4-(phenylacetyl)-11-azatricyclo[6.2.1.0˜2,7˜]undeca-2,4,6-trien-11-yl]ethyl}cyclohexyl)urea;
3-(2-{trans-4-[({[(1R)-2-hydroxy-1-methyl-2,2-diphenylethyl]amino}carbonyl)amino]cyclohexyl}ethyl)-3-methyl-7-(2-methylpropanoyl)-2,3,4,5-tetrahydro-1H-3-benzazepinium iodide;
Ethyl 1-(2-{trans-4-[({[(1S)-2-hydroxy-1-methyl-2,2-diphenylethyl]amino}carbonyl)amino]cyclohexyl}ethyl)-3-piperidinecarboxylate;
Lithium 1-(2-{trans-4-[({[(1S)-2-hydroxy-1-methyl-2,2-diphenylethyl]amino}carbonyl)amino]cyclohexyl}ethyl)-3-piperidinecarboxylate;
N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-2-methylpropyl}urea;
N-(trans-4-{2-[(1R,8S)-11-azatricyclo[6.2.1.0˜2,7˜]undeca-2,4,6-trien-11-yl]ethyl}cyclohexyl)-N′-{(1R)-1-[hydroxy(diphenyl)methyl]-2-methylpropyl}urea;
N-(trans-4-{2-[(1R,8S)-11-azatricyclo[6.2.1.0˜2,7˜]undeca-2,4,6-trien-11-yl]ethyl}cyclohexyl)-N′-{(1R)-1-[hydroxy(diphenyl)methyl]-3-methylbutyl}urea;
N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-3-methylbutyl}urea;
N-(trans-4-{2-[(1R,8S)-11-azatricyclo[6.2.1.0˜2,7˜]undeca-2,4,6-trien-11-yl]ethyl}cyclohexyl)-N′-[(1S)-2-hydroxy-1,2,2-triphenylethyl]urea;
N-(trans-4-{2-[(1R,8S)-11-azatricyclo[6.2.1.0˜2,7˜]undeca-2,4,6-trien-11-yl]ethyl}cyclohexyl)-N′-[(1R)-2-hydroxy-2,2-diphenyl-1-(phenylmethyl)ethyl]urea;
(2R)—N-(trans-4-{2-[(1R,8S)-11-azatricyclo[6.2.1.0˜2,7˜]undeca-2,4,6-trien-11-yl]ethyl}cyclohexyl)-2-[hydroxy(diphenyl)methyl-1-pyrrolidinecarboxamide;
(2S)—N-(trans-4-{2-[(1R,8S)-11-azatricyclo[6.2.1.0˜2,7˜]undeca-2,4,6-trien-11-yl]ethyl}cyclohexyl)-2-[hydroxy(diphenyl)methyl]-1-pyrrolidinecarboxamide;
1-dimethylethyl N-(2-{4-[({[(1S)-2-hydroxy-1-methyl-2,2-diphenylethyl]amino}carbonyl)amino]cyclohexyl}ethyl)-N-methylglycinate;
1-[(1S)-2-hydroxy-1-methyl-2,2-diphenylethyl]-3-(4-{2-[(1S,3R,5R,7S)-tricyclo[3.3.1.1^{3,7}]dec-1-ylamino]ethyl}cyclohexyl)urea;
1-[(1R)-2-hydroxy-1-methyl-2,2-diphenylethyl]-3-(trans-4-{2-[(phenylmethyl)amino]ethyl}cyclohexyl)urea;
1-[(1R)-2-hydroxy-1-methyl-2,2-diphenylethyl]-3-[trans-4-(2-{[(1R)-1-phenylethyl]amino}ethyl)cyclohexyl]urea;
(1R,5S)-8-(2-{4-[({[(1S)-2-hydroxy-1-methyl-2,2-diphenylethyl]amino}carbonyl)amino]cyclohexyl}ethyl)-3-[(3-hydroxy-2-phenylpropanoyl)oxy]-8-methyl-8-azoniabicyclo[3.2.1]octane iodide;
1-(trans-4-{2-[(1R)-2,3-dihydro-1H-inden-1-ylamino]ethyl}cyclohexyl)-3-[(1R)-2-hydroxy-1-methyl-2,2-diphenylethyl]urea trifluoroacetate (salt);
1-(trans-4-{2-[(1S)-2,3-dihydro-1H-inden-1-ylamino]ethyl}cyclohexyl)-3-[(1R)-2-hydroxy-1-methyl-2,2-diphenylethyl]urea;
(1R,5S)-8-(2-{trans-4-[({[(1R)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
1-(trans-4-{2-[(diphenylmethyl)amino]ethyl}cyclohexyl)-3-[(1R)-2-hydroxy-1-methyl-2,2-diphenylethyl]urea;
1-[(1R)-2-hydroxy-1-methyl-2,2-diphenylethyl]-3-[trans-4-(2-{[(1S)-1-(1-naphthalenyl)ethyl]amino}ethyl)cyclohexyl]urea;
1-[trans-4-(2-{[(1S,2R)-2-hydroxy-2,3-dihydro-1H-inden-1-yl]amino}ethyl)cyclohexyl]-3-[(1R)-2-hydroxy-1-methyl-2,2-diphenylethyl]urea;
1-{(1S)-1-[hydroxy(diphenyl)methyl]-2-methylpropyl}-3-[trans-4-(2-{[(1R)-1-phenylethyl]amino}ethyl)cyclohexyl]urea;
1-{(1R)-1-[hydroxy(diphenyl)methyl]-2-methylpropyl}-3-[trans-4-(2-{[(1R)-1-phenylethyl]amino}ethyl)cyclohexyl]urea;
1-{(1R)-1-[hydroxy(diphenyl)methyl]-3-methylbutyl)-3-[trans-4-(2-([(1R)-1-phenylethyl]amino}ethyl)cyclohexyl]urea;
1-{(1S)-1-[hydroxy(diphenyl)methyl]-3-methylbutyl}-3-[trans-4-(2-{([(1R)-1-phenylethyl]amino}ethyl)cyclohexyl]urea;
1-[(1S)-2-hydroxy-1,2,2-triphenylethyl]-3-[trans-4-(2-{[(1R)-1-phenylethyl]amino}ethyl)cyclohexyl]urea;
1-[(1R)-2-hydroxy-2,2-diphenyl-1-phenylmethyl)ethyl]-3-[trans-4-(2-{([(1R)-1-phenylethyl]amino}ethyl)cyclohexyl]urea;
(2R)-2-[hydroxy(diphenyl)methyl]-N-[trans-4-(2-{[(1R)-1-phenylethyl]amino}ethyl)cyclohexyl]-1-pyrrolidinecarboxamide;
(2S)-2-[hydroxy(diphenyl)methyl]-N-[trans-4-(2-{[(1R)-1-phenylethyl]amino}ethyl)cyclohexyl]-1-pyrrolidinecarboxamide;
Ethyl 1-(2-{trans-4-[({[(1R)-2-hydroxy-1-methyl-2,2-diphenylethyl]amino}carbonyl)amino]cyclohexyl}ethyl)-3-piperidinecarboxylate;
1-(2-{trans-4-[({[(1R)-2-hydroxy-1-methyl-2,2-diphenylethyl]amino}carbonyl)amino]cyclohexyl}ethyl)-3-piperidinecarboxylic acid trifluoroacetate (salt);
1-{trans-4-[2-(9H-fluoren-9-ylamino)ethyl]cyclohexyl}-3-[(1R)-2-hydroxy-1-methyl-2,2-diphenylethyl]urea;
1-[trans-4-(2-{[(1R,2S)-2-hydroxy-2,3-dihydro-1H-inden-1-yl]amino}ethyl)cyclohexyl]-3-[(1R)-2-hydroxy-1-methyl-2,2-diphenylethyl]urea;
1-[(1R)-2-hydroxy-1-methyl-2,2-diphenylethyl]-3-[trans-4-(2-{[(1R)-2-hydroxy-1-phenylethyl]amino}ethyl)cyclohexyl]urea;
1-[(1R)-2-hydroxy-1-methyl-2,2-diphenylethyl]-3-[trans-4-(2-{[(1S)-2-hydroxy-1-phenylethyl]amino}ethyl)cyclohexyl]urea;
(1R,5S)-8-[2-(trans-4-{[({(1S)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[({(1R)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[({(1R)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
(1R,5S)-8-[2-trans-4{[({(1S)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
(1R,5S)-8-(2-{trans-4-[({[(1S)-2-hydroxy-1,2,2-triphenylethyl]amino}carbonyl)amino]cyclohexyl}ethyl)-8-azabicyclo[3.2.1]oct-3-yl 3-hydroxy-2-phenylpropanoate;
(1R,5S)-8-(2-{trans-4-[({[(1R)-2-hydroxy-2,2-diphenyl-1-(phenylmethyl)ethyl]amino}carbonyl)amino]cyclohexyl}ethyl)-8-azabicyclo[3.2.1]oct-3-yl 3-hydroxy-2-phenylpropanoate;
1-[(1R)-2-hydroxy-1-methyl-2,2-diphenylethyl]-3-(4-trans-4-{2-[(1S)-1,2,3,4-tetrahydro-1-naphthalenylamino]ethyl}cyclohexyl)urea;
1-[(1R)-2-hydroxy-1-methyl-2,2-diphenylethyl]-3-trans-4-{2-[(1R)-1,2,3,4-tetrahydro-1-naphthalenylamino]ethyl}cyclohexyl)urea;
1-[(1R)-2-hydroxy-1-methyl-2,2-diphenylethyl]-3-[trans-4-(2-[{(1R)-1-(2-naphthalenyl)ethyl]amino}ethyl)cyclohexyl]urea;
1-[(1R)-2-hydroxy-1-methyl-2,2-diphenylethyl]-3-[trans-4-(2-{[(1S)-2-(4-methylphenyl)-1-phenylethyl]amino}ethyl)cyclohexyl]urea;
1-[(1R)-2-hydroxy-1-methyl-2,2-diphenylethyl]-3-(trans-4-{2-[(1-methyl-1-phenylethyl)amino]ethyl}cyclohexyl)urea;
1-(trans-4-{2-[(1R,8S)-11-azatricyclo[6.2.1.0^{2,7}]undeca-2,4,6-trien-11-yl]ethyl}cyclohexyl)-3-(2-cyano-1-methyl-2,2-diphenylethyl)urea;
1-(trans-4-{2-[(1R,8S)-11-azatricyclo[6.2.1.0^{2,7}]undeca-2,4,6-trien-11-yl]ethyl}cyclohexyl)-3-[(1S)-2-cyano-1-methyl-2,2-diphenylethyl]urea;
1-[(1S)-2-cyano-1-methyl-2,2-diphenylethyl]-3-{4-[2-(3-phenyl-1-piperidinyl)ethyl]cyclohexyl}urea;
1-[(1S)-2-cyano-1-methyl-2,2-diphenylethyl]-3-{4-[2-(2-phenyl-1-pyrrolidinyl)ethyl]cyclohexyl}urea;
1-[(1S)-2-cyano-1-methyl-2,2-diphenylethyl]-3-{trans-4-[2-(3-phenyl-1-pyrrolidinyl)ethyl]cyclohexyl}urea;
Ethyl 1-(2-{trans-4-[({[(1S)-2-cyano-1-methyl-2,2-diphenylethyl]amino}carbonyl)amino]cyclohexyl}ethyl)-3-piperidinecarboxylate;
1-[(1S)-2-cyano-1-methyl-2,2-diphenylethyl]-3-[trans-4-(2-{[(1R)-1-phenylethyl]amino}ethyl)cyclohexyl]urea;
(1R,5R)-8-(2-{trans-4-[({[(1S)-2-cyano-1-methyl-2,2-diphenylethyl]amino}carbonyl)amino]cyclohexyl}ethyl)-1,5-dimethyl-8-azabicyclo[3.2.1]oct-3-yl 3-hydroxy-2-phenylpropanoate;
1-(trans-4-{2-[(1R,8S)-11-azatricyclo[6.2.1.0^{2,7}]undeca-2,4,6-trien-11-yl]ethyl}cyclohexyl)-3-{(1S)-1[cyano(diphenyl)methyl]-3-methylbutyl}urea;
(1S,5S)-8-[2-(trans-4-{[({(1S)-1-[cyano(diphenyl)methyl]-3-methylbutyl}amino)carbonyl]amino}cyclohexyl)ethyl]-8-azabicyclo[3.2.1]oct-3-yl 3-hydroxy-2-phenylpropanoate; and
Ethyl 1-[2-(trans-4-{[({(1S)-1-[cyano(diphenyl)methyl]-3-methylbutyl}amino)carbonyl]amino}cyclohexyl)ethyl]-3-piperidinecarboxylate;
   and pharmaceutically acceptable salts thereof.

Preferred compounds of the present invention include:
(1R,5S)-8-(2-{trans-4-[({[(1S)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
(1R,5S)-8-[2-(trans-4-{[({(1R)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
N-(trans-4-{2-[(1R,8S)-11-azatricyclo[6.2.1.0˜2,7˜]undeca-2,4,6-trien-11-yl]ethyl}cyclohexyl)-N′-{(1R)-1-[hydroxy(diphenyl)methyl]-3-methylbutyl}urea;
Ethyl 1-(2-{trans-4-[({[(1S)-2-hydroxy-1-methyl-2,2-diphenylethyl]amino}carbonyl)amino]cyclohexyl}ethyl)-3-piperidinecarboxylate;
N-[(1R)-2-hydroxy-1-methyl-2,2-diphenylethyl]-N′-trans-4-{2-[7-(2-methylpropanoyl)-1,2,4,5-tetrahydro-3H-3-benzazepin-3-yl]ethyl}cyclohexyl)urea;
Ethyl 1-[2-(trans-4-{[({1S)-1-[cyano(diphenyl)methyl]-3-methylbutyl}amino)carbonyl]amino}cyclohexyl)ethyl]-3-piperidinecarboxylate′;
N-trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-3-methylbutyl}urea;
N-(trans-4-{2-[(1R,8S)-11-azatricyclo[6.2.1.0˜2,7˜]undeca-2,4,6-trien-11-yl]ethyl}cyclohexyl)-N′-[(1R)-2-hydroxy-2,2-diphenyl-1-(phenylmethyl)ethyl]urea;
1-(trans-4-{2-[(1S)-2,3-dihydro-1H-inden-1-ylamino]ethyl}cyclohexyl)-3-[(1R)-2-hydroxy-1-methyl-2,2-diphenylethyl]urea;
(1R,5S)-8-(2{trans-4-[({[(1R)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
(1R,5S)-8-(2-{trans-4-[({[(1R)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
(1S,5S)-8-[2-(trans-4-{[({(1S)-1-[cyano(diphenyl)methyl)-3-methylbutyl}amino)carbonyl]amino}cyclohexyl)ethyl]-8-azabicyclo[3.2.1]oct-3-yl 3-hydroxy-2-phenylpropanoate;
N-(trans-4-{2-[(1R,8S)-11-azatricyclo[6.2.1.0˜2,7˜]undeca-2,4,6-trien-11-yl]ethyl}cyclohexyl)-N′-[(1S)-2-hydroxy-1,2,2-triphenylethyl]urea;
N-(trans-4-{2-[(1R,8S)-1-azatricyclo[6.2.1.0˜2,7˜]undeca-2,4,6-trien-11-ylethyl}cyclohexyl)-N′-{(1R)-1-[hydroxy(diphenyl)methyl]-2-methylpropyl}urea;
N-[(1S)-2-hydroxy-1-methyl-2,2-diphenylethyl]-N′-(trans-4-{2-[7-(2-methylpropanoyl)-1,2,4,5-tetrahydro-3H-3-benzazepin-3-yl]ethyl}cyclohexyl)urea;
Ethyl 1-(2-{trans-4-[({[(1S)-2-cyano-1-methyl-2,2-diphenylethyl]amino}carbonyl)amino]cyclohexyl}ethyl)-3-piperidinecarboxylate;
1-[(1R)-2-hydroxy-1 methyl-2,2-diphenylethyl]-3-trans-4-{2-[(1-methyl-1-phenylethyl)amino]ethyl}cyclohexyl)urea;
(1R,5S)-8-(2-{4-[({[(1S)-2-hydroxy-1-methyl-2,2-diphenylethyl]amino}carbonyl)amino]cyclohexyl}ethyl)-3-[(3-hydroxy-2-phenylpropanoyl)oxy]-8-methyl-8-azoniabicyclo[3.2.1]octane iodide;
N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-2-methylpropyl}urea;
(2R)—N-(trans-4-{2-[(1R,8S)-11-azatricyclo[6.2.1.0˜2,7˜]undeca-2,4,6-trien-11-yl]ethyl}cyclohexyl)-2-[hydroxy(diphenyl)methyl]-1-pyrrolidinecarboxamide;
N-[(1R)-2-hydroxy-1-methyl-2,2-diphenylethyl]-N′-trans-{4-[2-(3-phenyl-1-piperidinyl)ethyl]cyclohexyl}urea;
1-{(1R)-1-[hydroxy(diphenyl)methyl]-3-methylbutyl}-3-[trans-4-(2-{[(1R)-1-phenylethyl]amino}ethyl)cyclohexyl]urea;
(1R,5R)-8-(2{trans-4-[({[(1S)-2-cyano-1-methyl-2,2-diphenylethyl]amino}carbonyl)amino]cyclohexyl}ethyl)-1,5-dimethyl-8-azabicyclo[3.2.1]oct-3-yl 3-hydroxy-2-phenylpropanoate;
N-[(1R)-2-hydroxy-1-methyl-2,2-diphenylethyl]-N′-{trans-4-[2-(3-phenyl-1-pyrrolidinyl)ethyl]cyclohexyl}urea;
9-(2-{trans-4-[({[(1R)-2-hydroxy-1-methyl-2,2-diphenylethyl]amino}carbonyl)amino]cyclohexyl}ethyl)-3-oxa-9-azatricyclo[3.3.1.0˜2,4˜]non-7-yl 3-hydroxy-2-phenylpropanoate;
1-[(1R)-2-hydroxy-2,2-diphenyl-1-(phenylmethyl)ethyl]-3-[trans-4-(2-{[(1R)-1-phenylethyl]amino}ethyl)cyclohexyl]urea;
1-(trans-4-{2-[(1R,8S)-11-azatricyclo[6.2.1.0^{2,7}]undeca-2,4,6-trien-11-yl]ethyl}cyclohexyl)-3-{(1S)-1-[cyano(diphenyl)methyl]-3-methylbutyl}urea;
1-(trans-4-{2-[(1R,8S)-11-azatricyclo[6.2.1.0^{2,7}]undeca-2,4,6-trien-11-yl]ethyl}cyclohexyl)-3-(2-cyano-1-methyl-2,2-diphenylethyl)urea;
1-trans-4-{2-[(1R,8S)-1-azatricyclo[6.2.1.0^{2,7}]undeca-2,4,6-trien-11-yl]ethyl}cyclohexyl)-3-(2-cyano-1-methyl-2,2-diphenylethyl)urea;
1-{(1S)-1-[hydroxy(diphenyl)methyl]-3-methylbutyl}-3-[trans-4-(2-{[(1R)-1-phenylethyl]amino}ethyl)cyclohexyl]urea;
′N-(trans-4-{2-[(1R,8S)-11-azatricyclo[6.2.1.0˜2,7˜]undeca-2,4,6-trien-11-yl]ethyl}cyclohexyl)-N′-[(1S)-2-hydroxy-1-methyl-2,2-diphenylethyl]urea;
1-[(1S)-2-hydroxy-1,2,2-triphenylethyl]-3-[trans-4-(2-{[(1R)-1-phenylethyl]amino}ethyl)cyclohexyl]urea;
(1R,2S,4R,5S)-9-(2-{trans-4-[({[(1S)-2-hydroxy-1-methyl-2,2-diphenylethyl]amino}carbonyl)amino]cyclohexyl}ethyl)-3-oxa-9-azatricyclo[3.3.1.0˜2,4˜]non-7-yl 3-hydroxy-2-phenylpropanoate;
N-(trans-4-{2-[(1R,8S)-4-azatricyclo[4.3.1.1˜3,8˜]undec-4-yl]ethyl}cyclohexyl)-N′-[(1R)-2-hydroxy-1-methyl-2,2-diphenylethyl]urea;
1-[(1R)-2-hydroxy-1-methyl-2,2-diphenylethyl]-3-[trans-4-(2-{[(1R)-1-phenylethyl]amino}ethyl)cyclohexyl]urea;
1-{(1S)-1-[hydroxy(diphenyl)methyl]-2-methylpropyl)-3-[trans-4-(2-([(1R)-1-phenylethyl]amino}ethyl)cyclohexyl]urea;
1{-(1R)-1-[hydroxy(diphenyl)methyl]-2-methylpropyl}-3-[trans-4-(2-{[(1R)-1-phenylethyl]amino}ethyl)cyclohexyl]urea;
N-[(1S)-2-hydroxy-1-methyl-2,2-diphenylethyl]-N′-{trans-4-[2-(3-phenyl-1-pyrrolidinyl)ethyl]cyclohexyl}urea;
1-[(1S)-2-cyano-1-methyl-2,2-diphenylethyl]-3-{4-[2-(2-phenyl-1-pyrrolidinyl)ethyl]cyclohexyl}urea;
1-(trans-4-{2-[(1R,8S)-11-azatricyclo[6.2.1.0²⁷]undeca-2,4,6-trien-11-yl]ethyl}cyclohexyl)-3-[(1S)-2-cyano-1-methyl-2,2-diphenylethyl]urea;
(1R,8S)-11-(2-{trans-4-[({[(1R)-2-hydroxy-1-methyl-2,2-diphenylethyl]amino}carbonyl)amino]cyclohexyl}ethyl)-11-methyl-11-azoniatricyclo[6.2.1.0˜2,7˜]undeca-2,4,6-triene iodide;
N-(trans-4-{2-[(1R,5S)-8-azabicyclo[3.2.1]oct-8-yl]ethyl}cyclohexyl)-N′-[(1S)-2-hydroxy-1-methyl-2,2-diphenylethyl]urea;
N-trans-4-{2-[(1R,5S)-8-azabicyclo[3.2.1]oct-8-yl]ethyl}cyclohexyl)-N′-[(1R)-2-hydroxy-1-methyl-2,2-diphenylethyl]urea;
N-{trans-4-[2-(3-azabicyclo[3.2.2]non-3-yl)ethyl]cyclohexyl}-N′-[(1S)-2-hydroxy-1-methyl-2,2-diphenylethyl]urea;
1-[(1S)-2-cyano-1-methyl-2,2-diphenylethyl]-3-{trans-4-[2-(3-phenyl-1-pyrrolidinyl)ethyl]cyclohexyl}urea;
(1R,8S)-11-(2-{trans-4-[({[(1S)-2-hydroxy-1-methyl-2,2-diphenylethyl]amino}carbonyl)amino]cyclohexyl}ethyl)-11-methyl-11-azoniatricyclo[6.2.1.0˜2,7˜]undeca-2,4,6-triene iodide;
1-[(1S)-2-hydroxy-1-methyl-2,2-diphenylethyl]-3-{trans-4-[2-(3-phenyl-1-piperidinyl)ethyl]cyclohexyl}urea;
2-(2{trans-4-[({[(1S)-2-hydroxy-1-methyl-2,2-diphenylethyl]amino}carbonyl)amino]cyclohexyl}ethyl)-2-methyl-2-azoniatricyclo[3.3.1.1˜3,7˜]decane iodide;
1-[(1R)-2-hydroxy-1-methyl-2,2-diphenylethyl]-3-[trans-4-(2-{[(1S)-2-hydroxy-1-phenylethyl]amino}ethyl)cyclohexyl]urea;
3-(2-{trans-4-[({[(1R)-2-hydroxy-1-methyl-2,2-diphenylethyl]amino}carbonyl)amino]cyclohexyl}ethyl)-3-methyl-7-(2-methylpropanoyl)-2,3,4,5-tetrahydro-1H-3-benzazepinium iodide;
N-(trans-4-{2-[(1s,4s)-7-azabicyclo[2.2.1]hept-7-yl]ethyl}cyclohexyl)-N′-[(1S)-2-hydroxy-1-methyl-2,2-diphenylethyl]urea;
N-(trans-4-{2-[(1s,4s)-7-azabicyclo[2.2.1]hept-7-yl]ethyl}cyclohexyl)-N′-[(1R)-2-hydroxy-1-methyl-2,2-diphenylethyl]urea;
1-[(1S)-2-hydroxy-1-methyl-2,2-diphenylethyl]-3-(4-{2-[(1S,3R,5R,7S)-tricyclo[3.3.1.1^{3,7}]dec-1-ylamino]ethyl}cyclohexyl)urea;
1,1-dimethylethyl N-(2-{4-[({[(1S)-2-hydroxy-1-methyl-2,2-diphenylethyl]amino}carbonyl)amino]cyclohexyl}ethyl)-N-methylglycinate;
N-[(1R)-2-hydroxy-1-methyl-2,2-diphenylethyl]-N′-{trans-4-[2-(2-phenyl-1-piperidinyl)ethyl]cyclohexyl}urea;
(1R,5S)-8-[2-(trans-4-{[({(1S)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[({(1S)-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
(1R,5S)-8-(2-{trans-4-[({[(1S)-2-hydroxy-1,2,2-triphenylethyl]amino}carbonyl)amino]cyclohexyl}ethyl)-8-azabicyclo[3.2.1]oct-3-yl 3-hydroxy-2-phenylpropanoate;
(1R,5S)-8-(2-{trans-4-[({[(1R)-2-hydroxy-2,2-diphenyl-1-(phenylmethyl)ethyl]amino}carbonyl)amino]cyclohexyl}ethyl)-8-azabicyclo[3.2.1]oct-3-yl 3-hydroxy-2-phenylpropanoate; and
(1R,5S)-8-[2-(trans-4-{[({(1R)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
   and pharmaceutically acceptable salts thereof.

Most preferred compounds include:
(1R,5S)-8-[2-(trans-4-{[({(1S)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[({(1S)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
(1R,5S)-8-(2-{trans-4-[({[(1S)-2-hydroxy-1,2,2-triphenylethyl]amino}carbonyl)amino]cyclohexyl}ethyl)-8-azabicyclo[3.2.1]oct-3-yl 3-hydroxy-2-phenylpropanoate;
(1R,5S)-8-(2-{trans-4-[({[(1R)-2-hydroxy-2,2-diphenyl-1-(phenylmethyl)ethyl]amino}carbonyl)amino]cyclohexyl}ethyl)-8-azabicyclo[3.2.1]oct-3-yl 3-hydroxy-2-phenylpropanoate; and
(1R,5S)-8-[2-(trans-4-{[({(1R)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate.

### METHODS OF PREPARATION

The compounds of Formula (I) may be obtained by utilizing synthetic procedures, some of which are illustrated in the Schemes below. The synthesis provided for these Schemes is applicable for producing compounds of formula (I) with a variety of different R1, R2, R3, Ar and Ar′. While the Schemes are shown with compounds only of Formula (I), this is merely for illustration purpose only.

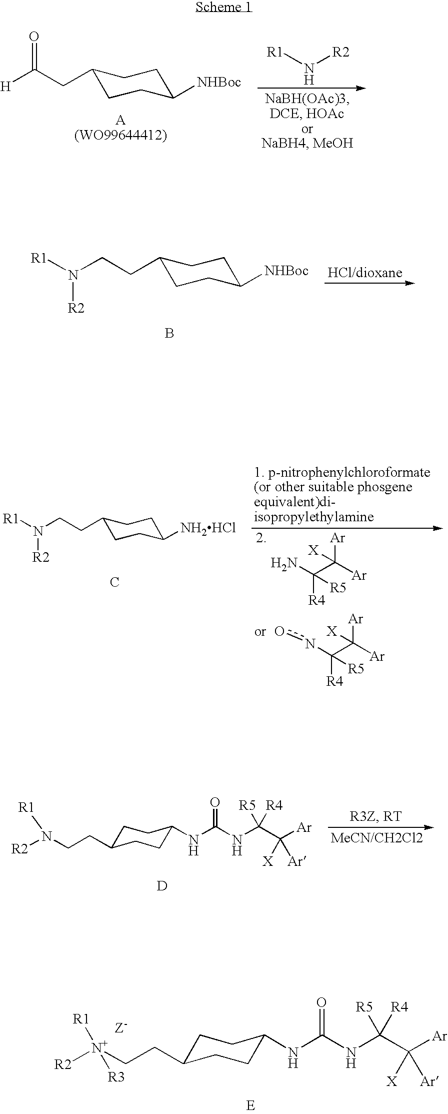

Scheme 1 illustrates one approach to the target molecules. Reductive amination of 1,1-dimethylethyl [4-(2-oxoethyl)cyclohexyl]carbamate (A) (prepared by known methods such as described in WO99644412) with amines afforded the mono-tert-butoxycarbonyl protected diamines (B). The procedure using NaBH(OAc)₃ was effective as described in the literature. (Abdel-Magid, A. F. et al, J. Org. Chem. 1996, 61, 3849) Also, as described in this reference, the alkylation of some of the primary

amines with aldehydes to form secondary amines, was more effective, in some cases, if a stepwise procedure was used involving imine formation in methanol, followed by reduction with NaBH₄. Removal of the tert-butoxycarbonyl protecting group from B with acid such as 4 M HCl in dioxane or neat trifluoroacetic acid affords the primary amine salts C. Stepwise reaction of the amines C with p-nitrophenylchloroformate, or other suitable phosgene equivalent (see Thavonekham, B. Synthesis 1997 1189-1194 and references therein) such as triphosgene, or bis(4-nitrophenyl)carbonate in the presence of an equivalent portion of tertiary amine base affords the activated carbamate (or similarly reactive) intermediate. Addition of the second amine to this intermediate in the presence of an additional equivalent portion of tertiary amine base affords the ureas (D). Alternatively reaction of the free base form of amines of general structure C and isocyanates will afford the desired products (D).

The quaternary salts (E) are prepared by reaction of the amine D with an excess of a suitably electrophilic alkyl halide, sulfonate or equivalent.

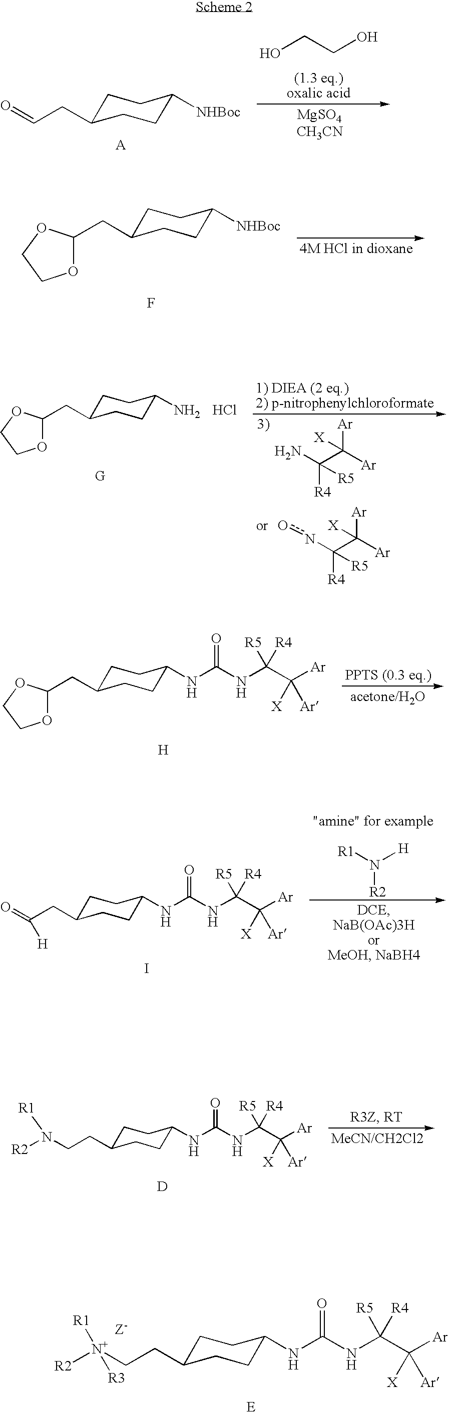

Scheme 2 illustrates an alternative approach to the target molecules. Acetal formation by any one of numerous standard conditions such as combining a mixture of aldehyde A, oxalic acid, ethylene glycol, solid anhydrous MgSO4 and CH3CN solvent, (Protective Groups in Organic Synthesis, 2^{nd} edition Greene, T. W.; Wuts, P.G.M. John Wiley & Sons, Inc New York 1991, p 188-195) affords the 1,3-dioxalane, F. Removal of the BOC amino protecting group with acids such as 4 M HCl in dioxane or neat trifluoroacetic acid affords the amine salts, G, which are reacted in a stepwise reaction with p-nitrophenylchloroformate, or other suitable phosgene equivalent (see Thavonekham, B. Synthesis 1997 1189-1194 and references therein) such as triphosgene, or bis(4-nitrophenyl)carbonate in the presence of a tertiary amine base to afford the activated carbamate (or similarly reactive) intermediate. Addition of the second amine to this intermediate in the presence of an additional equivalent of tertiary amine base affords the ureas (H). Alternatively reaction of the free base form of amines of general structure G and isocyanates will afford the desired products. Hydrolysis of the dioxalane in dilute aqueous acid containing an organic cosolvent, for example with pyridinium para-toluenesulfonic acid in aqueous acetone, affords aldehydes 1. Reductive amination of aldehydes I with amines afforded the target bases D. The procedure using NaBH(OAc)₃ was effective as described in the literature. (Abdel-Magid, A. F. et al, J. Org. Chem. 1996, 61, 3849) Also, as described in this reference, the alkylation of some of the primary amines with aldehydes to form secondary amines, was more effective, in some cases, if a stepwise procedure was used involving imine formation in methanol, followed by reduction with NaBH₄.

The quaternary salts (E) are prepared by reaction of the amine D with an excess of a suitably electrophilic alkyl halide, sulfonate or

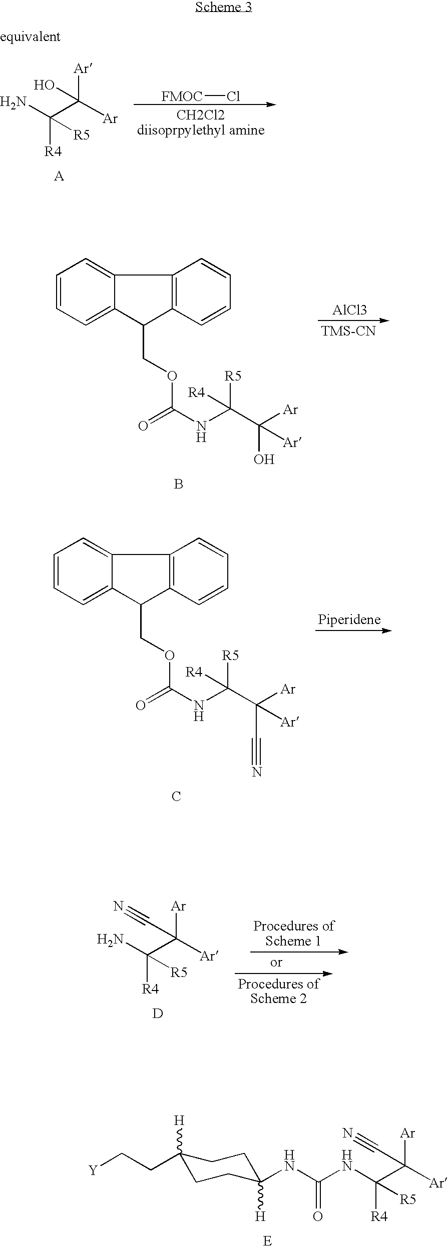

A procedure for conversion of the hydroxyethyl amines to cyanoethylamines and subsequent unsymmetrical urea formation is illustrated in Scheme 3. The FMOC protected amine B can be treated with TMS-CN in the presence of a Lewis acid catalyst such as AlCl₃ to afford the tertiary nitrile C. After deblocking to afford the cyano amine D, conversion to products E proceeds by the same methods as described in Schemes 1 and 2. Alternatively, the hydroxyl products, D, from Scheme 2 may be treated directly with AlCl3, and TMS-CN to afford the final products E in Scheme 3 in one step.

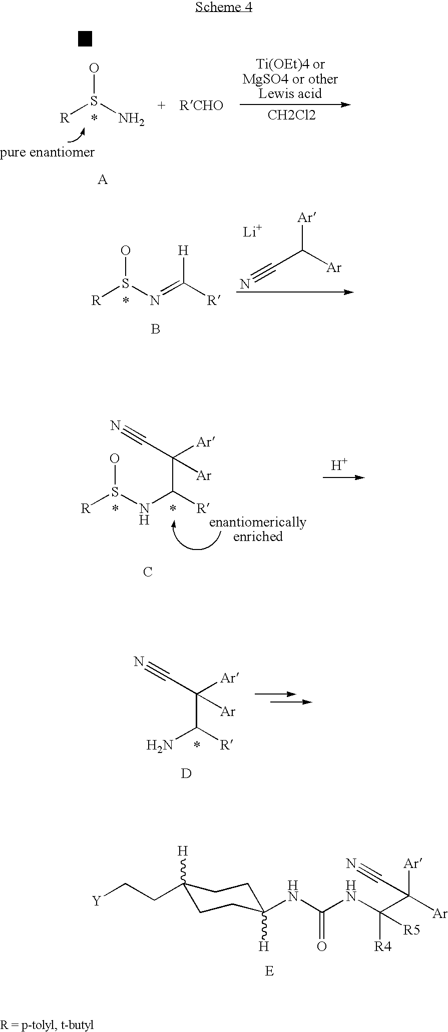

An alternative procedure for direct synthesis of the cyano amine analogs is depicted in Scheme 4. These methods are based on procedures developed by Davies and co-workers (Davies, F Zhou, P; Chen, B-C Chem Soc Rev 1998 27, 13-18). Thus a chiral sulfonamide A such as (S)-(+)-p-toluenesulfinamide (commercially available from Sigma-Aldrich) is reacted with aldehydes in the presence of a suitable Lewis acid dehydrating agent such as Ti(OEt)₄ by the methods described by Davis et al (Davis, F Zhang, Y; Andemichael, Y; Fang, T; Fanelli, D L; Zhang, H J Org Chem 1999 64, 1403-1406.) to afford enantiomerically pure sulfinimines B. These were reacted with the anions of diaryl acetonitriles to afford the diasteromerically enriched cyano sulfinamides C. The purity of intermediates can readily by ascertained by nmr. Furthermore, if necessary, further purification can be achieved by silica chromatography or crystallization. Removal of the chiral auxiliary as described by Davis and co-workers affords the cyano amines D which are converted to compounds of Formula (I) by the methods depicted in Schemes 1 and 2.

### SYNTHETIC EXAMPLES

### General Methods

The invention will now be described by reference to the following examples which are merely illustrative and are not to be construed as a limitation of the scope of the present invention. All temperatures are given in degrees centigrade, all solvents are highest available purity and all reactions run under anhyd conditions in an Ar atmosphere where necessary. Dry THF was obtained by distillation from sodium benzophenone ketyl. Where needed, other dry solvents were the Aldrich anhydrous solvents in Sure/Seal® bottles or the equivalent.

Mass spectra were run on an open access LC-MS system using electrospray ionization. LC conditions: 4.5% to 90% CH₃CN (0.02% TFA) in 3.2 min with a 0.4 min hold and 1.4 min re-equilibration; detection by MS, UV at 214 nm, and a light scattering-detector (ELS). Column: 1×40 mm Aquasil (C18). MH+ denotes the protonated molecular ion. Retention times (tR) are recorded in minutes for the specified LC conditions above.

¹H-NMR (hereinafter “NMR”) spectra were recorded at 400 MHz using a Bruker AM 400 spectrometer or a Bruker AVANCE 400. Multiplicities indicated are: s=singlet, d=doublet, t=triplet, q=quartet, m=multiplet and br indicates a broad signal.

“Gilson hplc” denotes injection of ca 50 mg of the final product in 500 uL of DMSO onto a 50×20 mm I. D. YMC CombiPrep ODS-A column at 20 mL/min with a 10 min gradient from 10% CH₃CN (0.1% TFA) to 90% CH₃CN (0.1% TFA) in H₂O (0.1% TFA) and a 2 min hold (unless otherwise stated). Flash chromatography was run over Merck Silica gel 60 (230-400 mesh) in solvent mixtures containing varying relative concentrations of dichloromethane and methanol, or EtOAc, and hexane, unless otherwise stated. Medium pressure chromatography was on ISCO RediSep® columns of the required size or the equivalent using an ISCO Combiflash® system or equivalent for pumping, detection, and peak collection. Chromatotron chromatography as has been previously described (Desai, H K; Joshi, B S; Panu, A M; Pelletier, S W J. Chromalogr. 1985 223-227.) was run on chromatotron plates available from Analtech, Wilmington Del., USA. Reverse phase cartridges were Varian MEGA BE, C18, 10 gm 60 mL cartridges or the equivalent. SCX cartridges were Applied Separations Spe-ed Benzenesulfonic SCX Cartridges cartridges or equivalent. Silica Spe-ed SPE cartridges were from Applied Separations.

Room temperature ═RT=23°; satd=saturated; aq=aqueous; NMP=1-methyl-2-pyrrolidinone; DCM=dichloromethane, NaBSA=sodium bis(trimethylsilyl)amide. Other abbreviations are as described in the ACS Style Guide (American Chemical Society, Washington, D.C., 1986).

### Example 1

### N-{4-[2-(dicyclohexylamino)ethyl]cyclohexyl}-N′-[(1R)-2-hydroxy-1-methyl-2,2-diphenylethyl]urea

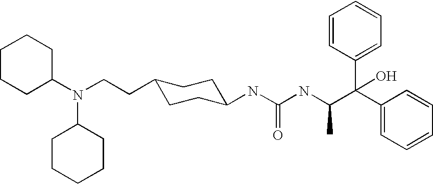

### 1a) Teri-butyl {4-[2-(dicyclohexylamino)ethyl]cyclohexyl}carbamate

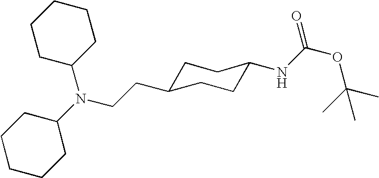

Tert-butyl [4-(2-oxoethyl)cyclohexyl]carbamate (WO99644412) (500 mg, 2.07 mmol) and dicyclohexylamine (400 mL, 2.07 mmol) were mixed in 1,2-dichloroethane (30 mL) for 30 mins., then treated with sodium triacetoxyborohydride (660 mg, 3.10 mmol). The mixture was stirred at 23° for 24 h. The reaction was quenched by adding aqueous sodium bicarbonate and dichloromethane. The organic layer was separated and the aqueous layer was extracted once with dichloromethane. The combined organic layers were dried (MgSO₄), filtered and concentrated, yielding 930 mg crude product as a dense liquid which is used directly in the next step. LCMS: m/z 407 (M+H) tR=1.86.

### 1b) [2-(4-aminocyclohexyl)ethyl]dicyclohexylamine

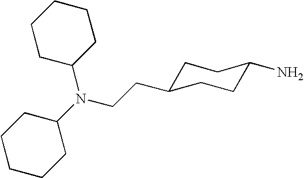

The product of the previous example (840 mg, 2.07 mmol) in dichloromrethane (20 mL) was added to trifluoroacetic acid (3 mL) and the mixture was stirred at 23° overnight. 2M NaOH was added, the organic layer was dried over MgSO4, filtered and concentrated to yield a white solid (720 mg) which is used without further purification. LCMS: m/z 307 (M+H)) tR=0.98.

### 1c) N-{4-[2-dicyclohexylamino)ethyl]cyclohexyl}-N′-[(1R)-2-hydroxy-1-methyl-2,2-diphenylethyl]urea ( )

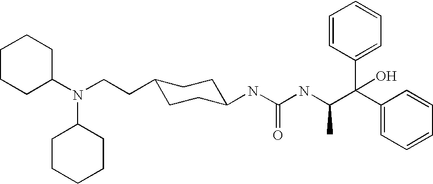

To the solution of [2-(4-aminocyclohexyl)ethyl]dicyclohexylamine (50 mg, 0.16 mmol) in chloroform (2 mL) was added 4-nitrophenyl chloroformate (33 mg, 0.16 mmol) in chloroform (2 mL) at 23°. After stirring for 1 h, (2S)-2-amino-1,1-diphenylpropan-1-ol (37 mg, 0.16 mmol) and diisopropylethyl amine (0.085 mL, 0.49 mmol) were added. The mixture was stirred overnight and then concentrated. Purify the concentrated mixture using Gilson to give the title product (15 mg).

LCMS: m/z 560 (M+H)) tR=2.19.

### Examples 2-5

Examples 2-5 were prepared following the general procedure of example 1 using the appropriate amine in example 1a to afford the title compounds depicted in Table 1. The amines were either commercially available or prepared by literature procedures

TABLE 1
Compounds directly prepared by the procedures of example 1

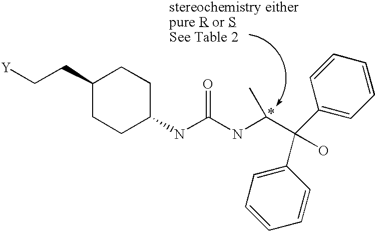

| | | | | Mass |
|---|---|---|---|---|
| | | | | Spec |
| example | Y | Chemical Name | Stereo | (Rf) |
| 2 | 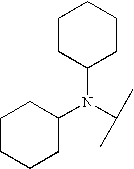 | N-{trans-4-[2- (dicyclohexylamino)ethyl]cyclohexyl}-N′- [(1S)-2-hydroxy-1-methyl-2,2- diphenylethyl]urea trifluoroacetate | S | 560 (2.27) |
| 3 | 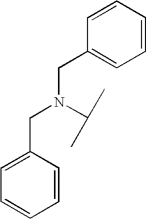 | N-(trans-4-{2- [bis(phenylmethyl)amino]ethyl}cyclohexyl)- N′-[(1R)-2-hydroxy-1-methyl-2,2- diphenylethyl]urea trifluoroacetate (salt) | R | 576 (2.14) |
| 4 | 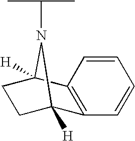 | N-(trans-4-{2-[(1R,8S)-11- azatricyclo[6.2.1.0~2,7~]undeca-2,4,6-trien- 11-yl]ethyl}cyclohexyl)-N′-1(1S)-2- hydroxy-1-methyl-2,2-diphenylethyl]urea | S | 524 |
| 5 | 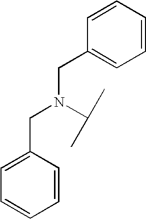 | N-(trans-4-{2- [bis(phenylmethyl)amino]ethyl}cyclohexyl)- N′-[(1S)-2-hydroxy-1-methyl-2,2- diphenylethyl]urea trifluoroacetate (salt) | S | 576 (2.12) |

### Example 6

### N-[(1R)-2-hydroxy-1-methyl-2,2-diphenylethyl]-N′-[trans-4-(2-{[(1R)-1-phenylethyl]amino}ethyl)cyclohexyl]urea [ ]

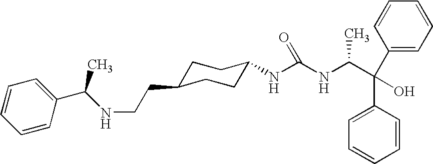

### 6a) 1,1-dimethylethyl [trans-4-(1,3-dioxolan-2-ylmethyl)cyclohexyl]carbamate

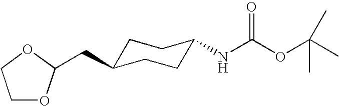

1,1-dimethylethyl [trans-4-(2-oxoethyl)cyclohexyl]carbamate (WO9964412) (24.1 g, 0.10 mol), anhydrous CH3CN (400 mL), oxalic acid, (1.2 g, 0.01 mol), ethylene glycol (24 mL), and anhydrous powdered MgSO4 (30 mL), were stirred together at 23° for 18 h. The reaction was filtered, diluted with EtOAc (1.5 L), and washed with satd aq NaHCO₃ (200 mL), H₂O (200 mL), and satd aq NaCl, dried (MgSO₄) and concentrated and dried in vacuo to afford 27.22 g (90%) of a white solid: ¹H NMR (400 MHz, CDCl₃) 4.90 (t, 3), 3.97 (m, 2), 3.85 (m, 2), 1.99 (m, 2), 1.86 (m, 2), 1.57 (m, 2), 1.45 (s, 9), 1.10 (m, 4).

### 6b) trans-4-(1,3-dioxolan-2-ylmethyl)cyclohexanamine hydrochloride

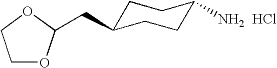

The product of the preceding example (27.2 g, 0.090 mol) and 4M HCl in dioxane (120 mL, 0.48 mol) were combined and a clear soln was obtained. The soln warmed slightly and began to bubble vigorously. After 10 min the warm reaction was cooled on an ice bath and a solid mass separated out. The mixture was diluted with dioxane (80 mL) and stirred 1 h, diluted again with Et₂O (400 mL) and filtered. The solid was washed with Et₂O (2×100 mL) and re-dried in vacuo to afford 20.1 g (100%). 1H NMR (400 MHz, CDCl3) 3.95 (m,2), 3.85 (m, 2), 3.03 (m, 1), 2.06-1.88 (m, 5), 1.51 (m, 3), 1.47 (m, 2), 1.06 (m, 2).

### 6c) N-[trans-4-(1,3-dioxolan-2-ylmethyl)cyclohexyl]-N′-[(1R)-2-hydroxy-1-methyl-2,2-diphenylethyl]urea

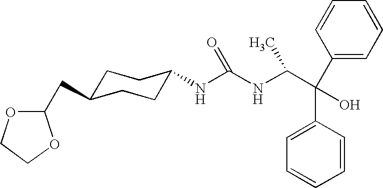

The product of Example 6b (4.41 g, 0.02 mol) in CH₃CN—CH₂Cl₂ (1:1) (200 mL) was treated with rapid stirring with diisopropylethylamine (7.00 mL, [5.16 g,] 0.04 mol). After rapid stirring for 30 minutes, this mixture was added in discrete increments to a solution of 4-nitrophenylchloroformate (4.02 g, 0.02 mol) in CH₃CN (100 mL) stirring at 0°. The resulting solution was stirred for 1 h at 0°. This mixture was in turn added to a 0° solution of (R)-(+)-2-Amino-1,1-diphenyl-1-propanol in CH₂Cl₂ (100 mL) and the resulting mixture was allowed to slowly come to room temperature overnight with stirring. LC/MS showed that the reaction was complete. Solution was evaporated to a small volume, then taken up in EtOAc. EtOAc solution was washed with 3×aq. Na₂CO₃, then with saturated aq NaCl and dried (Na₂SO₄), evaporation gave 9.5 g of a light yellow crude product. This was purified on silica column: (silica gel, step gradient, 0-4%, MeOH(CH₂Cl₂) to afford the title compound as a glassy solid Wt. 8.04 g (92%) LCMS: m/z 439 (M+H)⁺, tR=2.14.

### 6d) N-[(1R)-2-hydroxy-1-methyl-2,2-diphenylethyl]-N′-[trans-4-(2-oxoethyl)cyclohexyl]urea

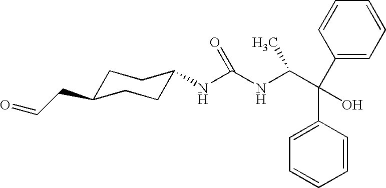

The product of Example 6c (3.31 g, 0.00756 mol) in acetone/water (8:2) 240 mL was gently refluxed with pyridinium p-toluenesulfonate (0.710 g, 0.00283 mol) over 72 h. LCMS indicated reaction was complete. Reaction was concentrated to near dryness then taken up in EtOAc; washed with sat. aq. NaHCO₃, then with H₂O, finally with saturated aq NaCl; dried (Na₂SO₄), then evaporated to afford a glassy colorless solid. Wt. 3.05 g (Quant.) LCMS: mtz 395 (M+H)⁺, tR=2.00.

### 6e) N-[(1R)-2-hydroxy-1-methyl-2,2-diphenylethyl]-N′-[trans-4-(2-{[(1R)-1-phenylethyl]amino)ethyl}cyclohexyl]urea

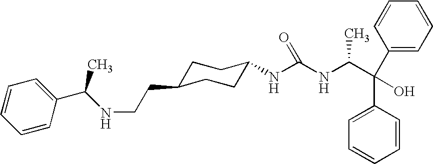

The product from Example 6d (0.070 g, 0.000185 mol) in 1,2-dichloroethane (5 mL) was treated with (1R)-1-Phenylethanamine (0.0182 g, 0.00015 mol) and with rapid stirring sodium triacetoxyborohydride (0.073 g, 0.000345 mol), was added followed by AcOH (44 mg, 0.00074 mol). Reaction continued to stir for an additional 18 h. LCMS showed reaction was complete. Reaction mixture was poured into sat. aq. NaHCO₃; EtOAc was added and the layers were separated; organic layer washed with sat aq. NaCl; dried (Na₂SO₄); evaporated to a residue which was taken up in CH₂Cl₂ and passed down a ‘SCX’ column; the column was then washed with MeOH (6 mL) and finally product was eluted with 2N NH₃ in MeOH (6 mL). [Fractions were monitored for purity with reversed phase TLC plates using 90:10 MeOH—H₂O and with LCMS]. Pure fractions combined and blown down under Argon to afford the title compound as a pale yellow glassy solid. Wt 61.6 mg (81.5%) LCMS: m/z 501 (M+H)⁺, tR=1.90.

The products of examples 7-52 depicted in Table 2 were prepared from the product of example 6d or the S enantiomer of the product of example 6d by reductive amination by the method of example 6e with the appropriate amine. The amines were either commercially available or prepared by literature methods.

TABLE 2
Compounds prepared directly from the product of Example 6d or the S
enantiomer of the product of Example 6d.

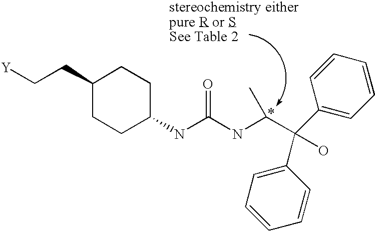

| | | | | Mass Spec |
|---|---|---|---|---|
| Ex- | | | | MH⁺ |
| ample | chemical name | Y | Stereo | (tR (min)) |
| 7 | N-(trans-4-{2-[(1R,5S)-3- azabicyclo[3.2.1]oct-3-yl] ethyl}cyclohexyl)-N′-[(1S)-2- hydroxy-1-methyl-2,2- diphenylethyl]urea | 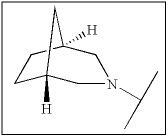 | S | 490 (1.75) |
| 8 | N-(trans-4-{2-[(1R,4S)-2- azabicyclo[2.2.1]hept-2-yl]ethyl} cyclohexyl)-N′-[(1R)-2- hydroxy-1-methyl-2,2- diphenylethyl]urea | 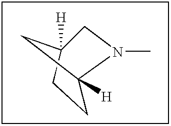 | R | 476 (1.64) |
| 9 | N-(trans-4-{2-[(1R,5S)-8- azabicyclo[3.2.1]oct-8-yl]ethyl} cyclohexyl)-N-[(1R)-2- hydroxy-1-methyl-2,2- diphenylethyl]urea | 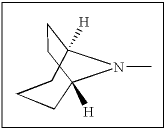 | R | 440 (1.75) |
| 10 | N-{trans-4-[2-(1,3-dihydro-2H- isoindol-2-yl)ethyl] cyclohexyl}-N-[(1R)-2- hydroxy-1-methyl-2,2- diphenylethyl]urea | 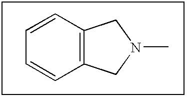 | R | 498 (1.83) |
| 11 | N-{trans-4-[2-(2,3-dihydro-1H- indol-1-yl)ethyl]cyclohexyl}- N′-[(1R)-2-hydroxy-1-methyl- 2,2-diphenylethyl]urea | 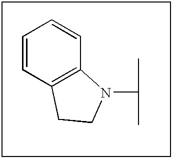 | R | 498 (2.01) |
| 12 | N-(trans-4-{2-[(1s,4s)-7- azabicyclo[2.2.1]hept-7- yl]ethyl} cyclohexyl)-N′-[(1R)-2- hydroxy-1-methyl-2,2- diphenylethyl]urea | 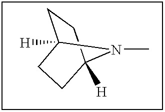 | R | 476 (1.65) |
| 13 | N-[(1R)-2-hydroxy-1-methyl- 2,2-diphenylethyl]- N′-(trans-4-{2-[7-(2- methylpropanoyl)-1,2,4,5- tetrahydro-3H-3-benzazepin-3- yl]ethyl}cyclohexyl)urea | 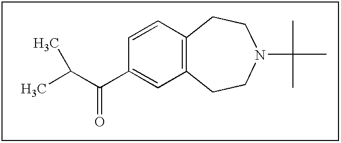 | R | 596 (1.98) |
| 14 | N-(trans-4-{2-[(1s,5s)-3- azabicyclo[3.2.2]non-3-yl]ethyl) cyclohexyl)-N′-[(1R)-2- hydroxy-1-methyl-2,2- diphenylethyl]urea | 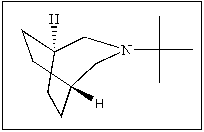 | R | 504 (1.83) |
| 15 | N-(trans-4-{2-[(1R,5S)-3- azabicyclo[3.2.1]oct-3-yl]ethyl} cyclohexyl)-N′-[(1R)-2- hydroxy-1-methyl-2,2- diphenylethyl]urea | 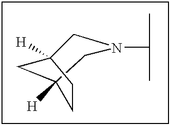 | R | 490 (1.32) |
| 16 | N-(trans-4-{2-[(1R,5S)-3- hydroxy-8-azabicyclo[3.2.1] oct-8-yl]ethyl}cyclohexyl)-N′- [(1R)-2-hydroxy-1-methyl-2,2- diphenylethyl]urea | 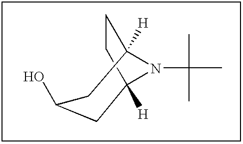 | R | 506 (1.59) |
| 17 | ethyl N-(2-{trans-4-[({[(1R)-2- hydroxy-1-methyl-2,2-diphenyl ethyl]amino}carbonyl)amino] cyclohexyl}ethyl)-N- methylglycinate | 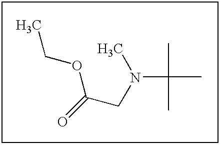 | R | 496 (1.63) |
| 18 | N-(trans-4-(2-[(IR,4S)-2- azabicyclo[2.2.1]hept-2-yl] ethyl}cyclohexyl)-N′-[(1S)-2- hydroxy-1-methyl-2,2- diphenylethyl]urea | 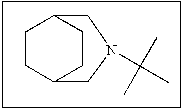 | S | 504 (1.99) |
| 19 | N-(trans-4-{2-[(1R,4S)-2- azabicyclo[2.2.1]hept-2-yl] ethyl}cyclohexyl)-N′-[(1S)-2- hydroxy-1-methyl-2,2- diphenylethyl]urea | 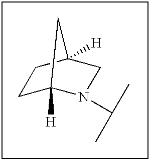 | S | 476 (1.73) |
| 20 | N-(trans-4-{2-[(1s,4s)-7- azabicyclo[2.2.1]hept-7-yl] ethyl}cyclohexyl)-N′-[(1S)-2- hydroxy-1-methyl-2,2- diphenylethyl]urea | 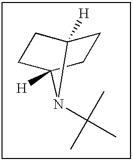 | S | 476 (1.70) |
| 21 | N-(trans-4-{2-[(1R,5S)-8- azabicyclo[3.2.1]oct-8-yl] ethyl}cyclohexyl)-N′-[(1S)-2- hydroxy-1-methyl-2,2- diphenylethyl]urea | 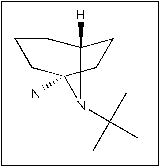 | S | 490 (1.84) |
| 22 | (1R,5S)-8-(2-{trans-4-[({[(1S)- 2-hydroxy-1-methyl-2,2- diphenylethyl]amino]carbonyl) amino}cyclohexyl}ethyl)-8- azabicyclo[3.2.1]oct-3-yl 3- hydroxy-2-phenylpropanoate | 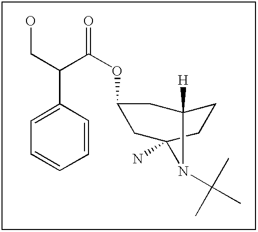 | S | 654 (1.99) |
| 23 | (1R,2S,4R,5S)-9-(2-{trans-4- [({[(1S)-2-hydroxy-1-methyl- 2,2- diphenylethyl]amino}carbonyl) amino]cyclohexyl}ethyl)-3-oxa- 9- azatricyclo[3.3.1.0~2,4~]non-7- yl 3-hydroxy-2- phenylpropanoate | 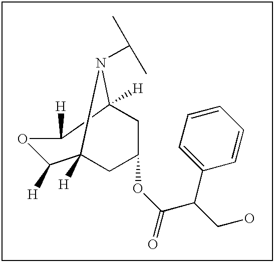 | S | 668 (1.87) |
| 24 | N-{trans-4-[2-(3,4-dihydro- 1(2H)-quinolinyl)ethyl] cyclohexyl}-N′-[(1S)-2- hydroxy-1-methyl-2,2- diphenylethyl]urea | 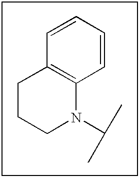 | S | 512 (2.31) |
| 25 | N-{trans-4-[2-(3,4-dihydro- 2(1H)-isoquinolinyl)ethyl] cyclohexyl}-N′-[(1S)-2- hydroxy-1-methyl-2,2- diphenylethyl]urea | 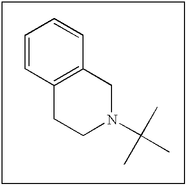 | S | 512 (1.87) |
| 26 | N-(trans-4-{2-[(1R,5S)-3- hydroxy-8-azabicyclo[3.2.1] oct-8-yl]ethyl}cyclohexyl)-N′- [(1S)-2-hydroxy-1-methyl-2,2- diphenylethyl]urea | 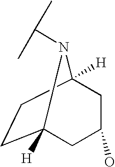 | S | 506 (1.72) |
| 27 | N-[(1S)-2-hydroxy-1-methyl- 2,2-diphenylethyl]-N′- {trans-4-[2-(2-phenyl-1- pyrrolidinyl)ethyl]cyclohexyl}urea | 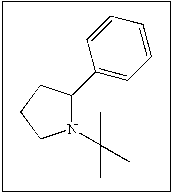 | S | 526 (1.99) |
| 28 | N-[(1S)-2-hydroxy-1-methyl- 2,2-diphenylethyl]-N′- {trans-4-[2-(4-phenyl-1- piperidinyl)ethyl]cyclohexyl}urea | 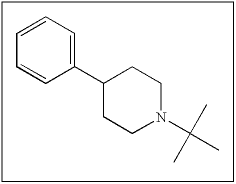 | S | 540 (2.02) |
| 29 | N-(trans-4-{2-[(1R,8S)-4- azatricyclo[4.3.1.1~3,8~] undec-4-yl]ethyl}cyclohexyl)- N′-[(1R)-2-hydroxy-1-methyl- 2,2-diphenylethyl]urea | 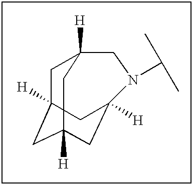 | R | 530 (2.09) |
| 30 | 1,1-dimethylethyl N-(2-{trans-4- [({[(1R)-2-hydroxy- 1-methyl-2,2- diphenylethyl]amino}carbonyl) amino]cyclohexyl}ethyl)-N- methylglycinate | 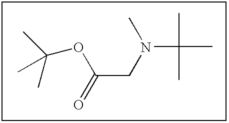 | R | 524 (1.96) |
| 31 | N-[(1R)-2-hydroxy-1-methyl- 2,2-diphenylethyl]- N′-{trans-4-[2-(2-phenyl-1- piperidinyl)ethyl]cyclohexyl}urea | 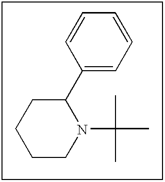 | R | 540 (2.07) |
| 32 | N-[(1R)-2-hydroxy-1-methyl- 2,2-diphenylethyl]- N′-(trans-4-{2- [methyl(phenylmethyl)amino] ethyl}cyclohexyl)urea | 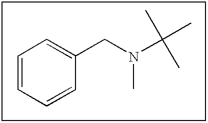 | R | 500 (1.94) |
| 33 | N-[(1R)-2-hydroxy-1-methyl- 2,2-diphenylethyl]-N′- {trans-4-[2-(2-phenyl-1- pyrrolidinyl)ethyl]cyclohexyl}urea | 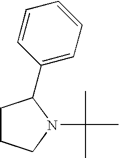 | R | 526 (2.01) |
| 34 | N-(2-{trans-4-[({[(1R)-2- hydroxy-1-methyl-2,2- diphenylethyl]amino}carbonyl) amino]cyclohexyl}ethyl)-N- methylglycine | 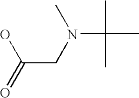 | R | 450 (1.65) |
| 35 | N-[(1R)-2-hydroxy-1-methyl- 2,2-diphenylethyl]- N′-{trans-4-[2-(3-phenyl-1- pyrrolidinyl)ethyl]cyclohexyl}urea | 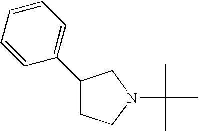 | R | 526 (2.04) |
| 36 | N-[(1R)-2-hydroxy-1-methyl-2,2- diphenylethyl]-N′- {trans-4-[2-(3-phenyl-1- piperidinyl)ethyl]cyclohexyl}urea | 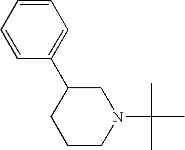 | R | 540 (2.12) |
| 37 | 9-(2-[trans-4-[({[(1R)-2- hydroxy-1-methyl-2,2- diphenylethyl]amino}carbonyl) amino]cyclohexyl}ethyl)-3-oxa- 9-azatricyclo[3.3.1.0~2,4~]non- 7-yl 3- hydroxy-2-phenylpropanoate | 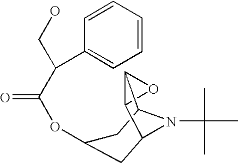 | R | 668 (1.99) |
| 38 | N-[(1S)-2-hydroxy-1-methyl- 2,2-diphenylethyl]-N′- (trans-4-{2-[(1R,5S)-3-hydroxy- 3-phenyl-8-azabicyclo[3.2.1]oct- 8-yl]ethyl}cyclohexyl)urea | 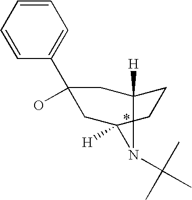 | S | 582 (2.00) |
| 39 | ethyl N-(2-{trans-4-[({[(1S)-2- hydroxy-1-methyl-2,2- diphenylethyl]amino}carbonyl) amino]cyclohexyl}ethyl)-N- methylglycinate | 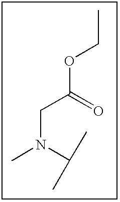 | S | 496 (1.78) |
| 40 | N-[(1S)-2-hydroxy-1-methyl- 2,2-diphenylethyl]-N′- (trans-4-{2-[7-(2- methylpropanoyl)-1,2,4,5- tetrahydro-3H-3-benzazepin-3- yl]ethyl}cyclohexyl)urea | 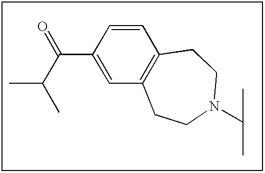 | S | 596 (1.99) |
| 41 | N-[(1S)-2-hydroxy-1-methyl- 2,2-diphenylethyl]-N′- {trans-4-[2-(3-phenyl-1- piperidinyl)ethyl]cyclohexyl}urea | 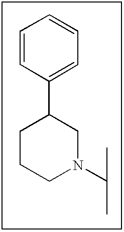 | S | 540 (2.07) |
| 42 | N-(trans-4-{2-[(1R,8S)-4- azatricyclo[4.3.1.1~3,8~] undec-4-yl]ethyl}cyclohexyl)- N′-[(1S)-2-hydroxy-1-methyl- 2,2-diphenylethyl]urea | 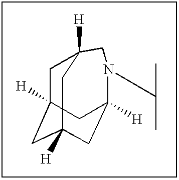 | S | 530 (2.04) |
| 43 | N-(trans-4-{2-[(1R,8S)-11- azatricyclo[6.2.1.0~2,7~] undeca-2,4,6-trien-11- yl]ethyl}cyclohexyl)-N′-[(1R)-2- hydroxy-1-methyl-2,2- diphenylethyl]urea | 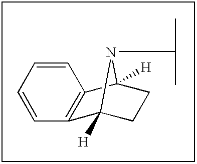 | R | 524 (1.93) |
| 44 | N-[(1S)-2-hydroxy-1-methyl- 2,2-diphenylethyl]-N′-{trans-4- [2-(3-phenylpyrrolidin-1- yl)ethyl]cyclohexyl}urea | 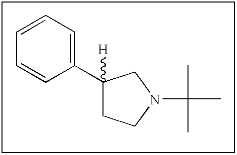 | S | 526 (1.94) |
| 45 | N-[(1S)-2-hydroxy-1-methyl- 2,2-diphenylethyl]-N′-{trans-4- [2-(2-phenylpiperidin-1- yl)ethyl]cyclohexyl}urea | 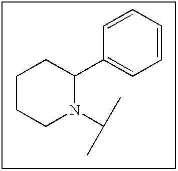 | S | 540 (2.03) |
| 46 | ethyl 1-(2-{trans-4-[({[(1S)-2- hydroxy-1-methyl-2,2- diphenylethyl]amino}carbonyl) amino]cyclohexyl}ethyl)-3- piperidinecarboxylate | 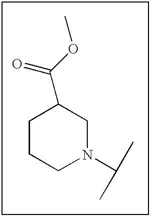 | S | 536 (1.75) |
| 47 | ethyl 1-(2-{trans-4-[({[(1R)-2- hydroxy-1-methyl-2,2- diphenylethyl]amino}carbonyl) amino]cyclohexyl}ethyl)-3- piperidinecarboxylate | 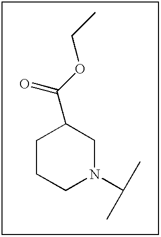 | R | 536 (1.72) |
| 48 | lithium 1-(2-{trans-4-[({[(1S)-2- hydroxy-1-methyl-2,2- diphenylethyl]amino}carbonyl) amino]cyclohexyl}ethyl)piperidine- 3-carboxylate | 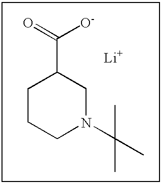 | S | 508 (1.59) |
| 49 | N-[(1R)-2-hydroxy-1-methyl- 2,2-diphenylethyl]-N′-(trans-4- {2-[(1S,4R)-6-(phenylacetyl)- 1,2,3,4-tetrahydro-1,4- epiminonaphthalen-9- yl]ethyl}cyclohexyl)urea | 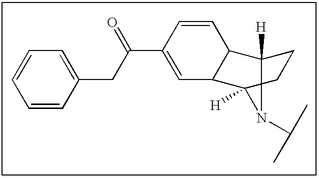 | R | 642 (2.06) |
| 50 | N-[(1R)-2-hydroxy-1-methyl- 2,2-diphenylethyl]-N′-{trans-4- [2-(tricyclo[3.3.1.1~3.7~]dec-1- ylamino)ethyl]cyclohexyl}urea | 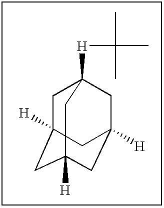 | R | 530 (1.91) |
| 51 | N-[(1R)-2-hydroxy-1-methyl- 2,2-diphenylethyl]-N′-(trans-4- {2- [(phenylmethyl)amino]ethyl} cyclohexyl)urea | 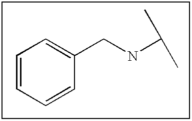 | R | 486 (1.74) |
| 52 | N-(trans-4-{2-[(1R)-2,3- dihydro-1H-inden-1- ylamino]ethyl}cyclohexyl)-N′- [(1R)-2-hydroxy-1-methyl-2,2- diphenylethyl]urea trifluoroacetate (salt) | 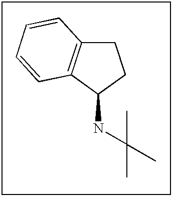 | R | 512 (1.88) |
| 53 | N-(trans-4-{2-[(1S)-2,3- dihydro-1H-inden-1- ylamino]ethyl}cyclohexyl)-N′- [(1R)-2-hydroxy-1-methyl-2,2- diphenylethyl]urea | 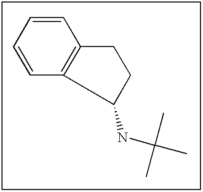 | R | 512 (1.91) |

The product of Example 48 is prepared from the product of example 46 by LiOH hydrolysis.

### Quaternary Salts

### Example 54

### (1R,8S)-11-(2-{trans-4-[({[(1S)-2-hydroxy-1-methyl-2,2-diphenylethyl]amino}carbonyl)amino]cyclohexyl}ethyl)-11-methyl-11-azoniatricyclo[6.2.1.0˜2,7˜-]undeca-2,4,6-triene iodide

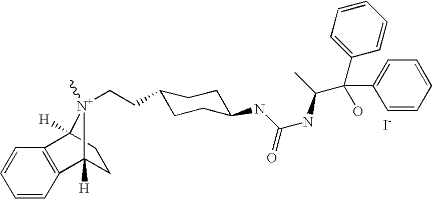

To a solution of the product of example 4 (32 mg, 0.06 mmol) in 3 mL CH₂Cl₂/CH₃CN (2:1) was added methyl iodide (19 μL, 0.30 mmol) at rt. LC-MS showed product without starting material after overnight and the concentrated mixture was purified by reverse-phase cartridge to give 25 mg desired quat salt. LCMS: m/z 538 (M+H)⁺, tR=1.81

The products of examples 55-58 depicted in Table 3 were prepared by the method of example 54 from the product of the appropriate example as specified in Table 3.

TABLE 3
Quaternary salts.

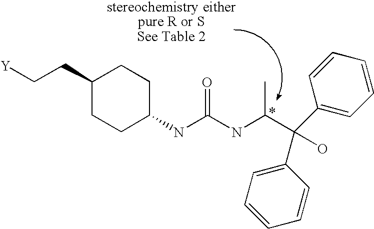

| | starting | | | | |
|---|---|---|---|---|---|
| | with | | | | |
| | the | | | | Mass |
| | product of | | | | Spec |
| Example | example | Y | Chemical Name | stereo | (tR) |
| 55 | 43 | 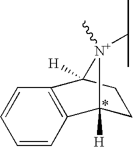 | (1R,8S)-11-(2-{trans-4- [({[(1R)-2-hydroxy-1- methyl-2,2- diphenylethyl]amino} carbonyl)amino]cyclohexyl} ethyl)-11-methyl-11- azoniatricyclo[6.2.1.0-2,7~] undeca-2,4,6-triene iodide | R | 538 (2.00) |
| 56 | 41 | 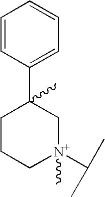 | 1-(2-{trans-4-[({[(1R)-2- hydroxy-1-methyl-2,2- diphenylethyl]amino} carbonyl)amino]cyclohexyl}ethyl)- 1,3-dimethyl-3- phenylpiperidinium iodide | R | 554 (2.03) |
| 57 | 42 | 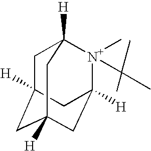 | 2-(2-{trans-4-[({[(1S)-2- hydroxy-1-methyl-2,2- diphenylethyl]amino} carbonyl)amino]cyclohexyl} ethyl)-2-methyl-2- azoniatricyclo[3.3.1.1~3,7~] decane iodide | S | 544 (1.99) |
| 58 | 13 | 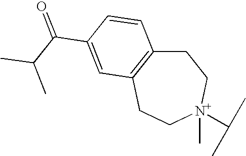 | 3-(2-{trans-4-[({[(1R)-2- hydroxy-1-methyl-2,2- diphenylethyl]amino} carbonyl)amino]cyclohexyl} ethyl)-3-methyl-7-(2- methylpropanoyl)-2,3,4,5- tetrahydro-1H-3- benzazepinium iodide | R | 611 (1.97) |
| 59 | 68 | 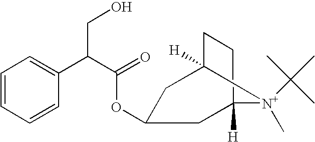 | (1R,5S)-8-(2-{4-[({[(1S)-2- hydroxy-1-methyl-2,2- diphenylethyl]amino} carbonyl)amino]cyclohexyl} ethyl)-3-[(3-hydroxy-2- phenylpropanoyl) oxy]-8-methyl-8- azoniabicyclo[3.2.1]octane iodide | S | |

### Example 60

### N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-2-methylpropyl}urea

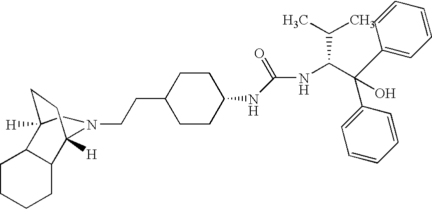

### 60a) N-[trans-4-(1,3-dioxolan-2-ylmethyl)cyclohexyl]-N′-{(1R)-1-[hydroxy(diphenyl)methyl]-2-methylpropyl}urea

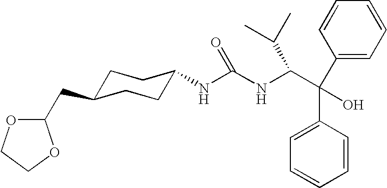

The product of example 6b (0.43 g, 0.00196 mol) was stirred rapidly at RT in CH₃CN—CH₂Cl₂ (1:1) (20 mL) with diisopropylethylamine [0.7 mL, 0.516 g, 0.004 mol]. After 30 minutes this was cooled to 0° and added at a moderate rate to a rapidly stirring solution of 4-nitrophenylchloroformate (0.394 g, 0.00196 mol) in CH₃CN (10 mL), at 0°; solution was stirred for 40 minutes. This mixture was, in turn, added, in one portion, to a rapidly stirring solution of (S)-(−)-2-amino-3-methyl-1,1-diphenyl-1-butanol (0.5 g, 0.00196 mol) in CH₂Cl₂ (10 mL)[dry] at 0°. Solution was stirred at 0° and warmed to room temperature overnight. LC/MS shows reaction is complete; reaction stripped to near dryness; taken up in EtOAc; solution washed with aq. Na₂CO₃ (3×100 mL), then with saturated aq NaCl, dried (Na₂SO₄), then solvent was evaporated to afford a glassy pale yellow foam. (0.9362 g). This was taken up in CH₂Cl₂ and applied to a chromatotron plate. Plate eluted with EtOAc-hexane (40:60) to afford the desired product as a glassy foam. (0.7490 g, 82%) LCMS: mtz 467 (M+H)⁺, tR=2.25.

### 60b) N-{(1R)-1-[hydroxy(diphenyl)methyl]-2-methylpropyl}-N′-[trans-4-(2-oxoethyl)cyclohexyl]urea

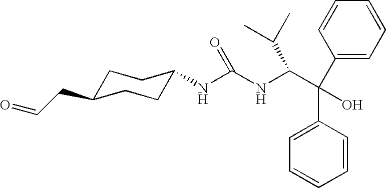

The product from Example 60a (0.7490 g, 0.0016 mol) in acetone —H₂O (8:2) (60 mL) with pyridinium p-toluenesulfonate (0.141 g, 0.00056 mol) was gently refluxed for 72 h, under Ar. LC/MS showed the reaction to be complete. Reaction was stripped to near dryness then taken up in EtOAc and washed in turn with satd aq NaHCO₃, H₂O, then satd aq NaCl; dried (Na₂SO4); then solvent was evaporated to afford the title compound as a glassy near colourless foam. Wt. 0.6627 g. (98%) LCMS: m/z 423 (M+H)⁺, tR=2.12.

### 60c) N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-2-methylpropyl}urea

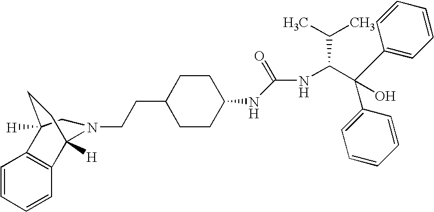

The product from Example 60b (0.078 g, 0.000185 mol) in 1,2-dichloroethane (5 mL) was treated with 11-azatricyclo[6.2.1.0^{2,7}]undeca-2,4,6-triene (0.022 g, 0.00015 mol) and with rapid stirring sodium triacetoxyborohydride (0.073 g, 0.000345 mol), was added followed by AcOH (44 mg, 0.00074 mol). Reaction continued to stir for an additional 18 h. LCMS showed reaction was complete. Reaction mixture was taken up into EtOAc; washed with satd aq NaHCO₃, then satd aq NaCl; dried (Na₂SO₄); evaporated to a residue which was taken up in CH₂Cl₂. The solution was passed down an SCX column which was washed with CH₂Cl₂ (5 mL), then with MeOH (8 mL). Product was eluted with 2N NH₃ in MeOH (6 mL). This latter eluent was evaporated to a glassy solid to afford the title compound as an off white solid Wt. 49.3 mg (48%) LCMS: m/z 553 (M+H)⁺, tR=2.05.

Intermediates A-H were prepared from the product of example 6b and the appropriate 1,1-diphenylamino-1-hydroxy alkyl amine (NRR′) by the method of example 6c and are depicted in Table 4.

TABLE 4
Intermediates A-H were prepared from the product of example 6b and the appropriate
1,1-diphenylamino-1-hydroxy alkyl amino (NRR′) by the method of example 6c.

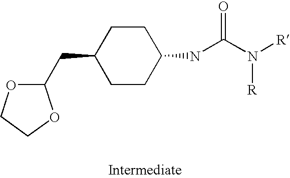

| | | | Mass Spec |
|---|---|---|---|
| Intermediate | Chemical name | NRR′ | (tR) |
| A | N-[trans-4-(1,3-dioxolan-2- ylmethyl)cyclohexyl]-N′-{(1R)-1- [hydroxy(diphenyl)methyl]-2- methylpropyl}urea | 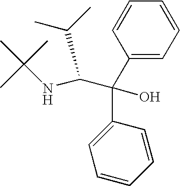 | 467.4 (2.25) |
| B | N-[trans-4-(1,3-dioxolan-2- ylmethyl)cyclohexyl]-N′-{(1S)-1- [hydroxy(diphenyl)methyl]-2- methylpropyl}urea | 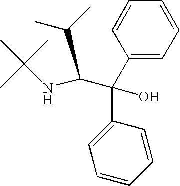 | 467.4 (2.30) |
| C | N-[trans-4-(1,3-dioxolan-2- ylmethyl)cyclohexyl]-N′-{(1R)-1- [hydroxy(diphenyl)methyl]-3- methylbutyl}urea | 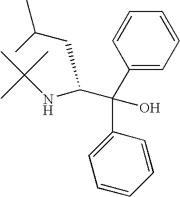 | 481.2 (2.38) |
| D | N-[trans-4-(1,3-dioxolan-2- ylmethyl)cyclohexyl]-N′-{(S)-1- [hydroxy(diphenyl)methyl]-3- methylbutyl}urea | 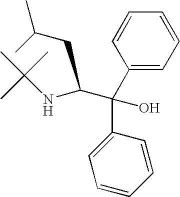 | 481.2 (2.39) |
| E | N-[trans-4-(1,3-dioxolan-2- ylmethyl)cyclohexyl]-N′-[(1S)-2- hydroxy-1,2,2-triphenylethyl]urea | 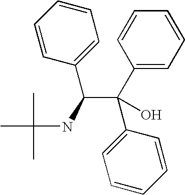 | 501.1 (2.38) |
| F | N-[trans-4-(1,3-dioxolan-2- ylmethyl)cyclohexyl]-N′-[(1R)-2- hydroxy-2,2-diphenyl-1- (phenylmethyl)ethyl]urea | 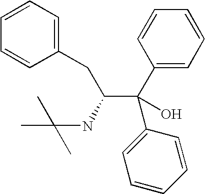 | 515.4 (2.42) |
| G | (2R)-N-[trans-4-(1,3-dioxolan-2- ylmethyl)cyclohexyl]-2- [hydroxy(diphenyl)methyl]-1- pyrrolidinecarboxamide | 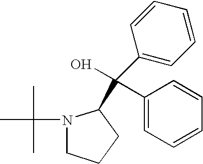 | 466.4 (2.18) |
| H | (2S)-N-[trans-4-(1,3-dioxolan-2- ylmethyl)cyclohexyl]-2- [hydroxy(diphenyl)methyl]-1- pyrrolidinecarboxamide | 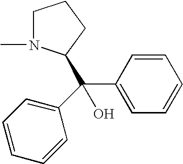 | 465.2 (2.40) |

TABLE 5
Examples 61-67 were made from the indicated 1,1-diaryl-1-hydroxy amino alcohol
intermediate from Table 4 by the methods of Example 60b and 60c.

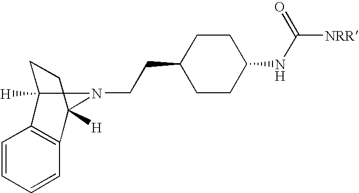

| | | | | |
|---|---|---|---|---|
| | | | | Mass |
| | NRR′ | | StereoHR | spec |
| example | (Intermediate) | chemical name | at R | (tR) |

| | | | | |
|---|---|---|---|---|
| 61 | 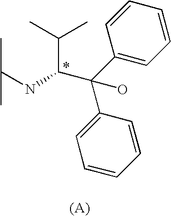 | N-(trans-4-{2-[(1R,8S)-11- azatricyclo[6.2.1.0~2,7~]undeca- 2,4,6-trien-11-yl]ethyl}cyclohexyl)- N′-{(1R)-1- [hydroxy(diphenyl)methyl]-2- methylpropyl}urea | R | 552.6 (2.00) |
| 62 | 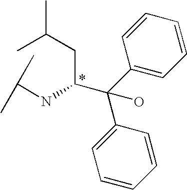 | N-(trans-4-{2-[(1R,8S)-11- azatricyclo[6.2.1.0~2,7~]undeca- 2,4,6-trien-11-yl]ethyl}cyclohexyl)- N′-{(1R)-1- [hydroxy(diphenyl)methyl]-2- methylbutyl}urea | R | 566.4 (2.05) |
| 63 | 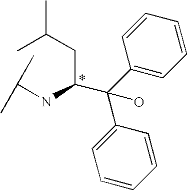 | N-(trans-4-{2-[(1R,8S)-11- azatricyclo[6.2.1.0~2,7~]undeca- 2,4,6-trien-11-yl]ethyl}cyclohexyl)- N′-{(1S)-1- [hydroxy(diphenyl)methyl]-3- methylbutyl}urea | S | 566.2 (2.14) |
| 64 | 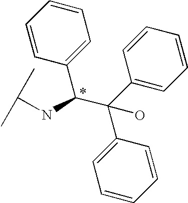 | N-(trans-4-{2-[(1R,8S)-11- azatricyclo[6.2.1.0~2,7~]undeca- 2,4,6-trien-11-yl]ethyl}cyclohexyl)- N′-[(1S)-2-hydroxy-1,2,2- triphenylethyl]urea | S | 586.4 (2.03) |
| 65 | 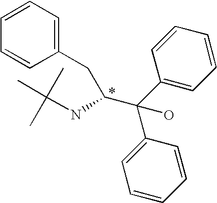 | N-(trans-4-{2-[(1R,8S)-11- azatricyclo[6.2.1.0~2,7~]undeca- 2,4,6-trien-11-yl]ethyl}cyclohexyl)- N′-[(1R)-2-hydroxy-2,2-diphenyl-1- (phenylmethyl)ethyl]urea | R | 600.6 (2.09) |
| 66 | 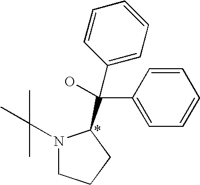 | (2R)-N-(trans-4-{2-[(1R,8S)-11- azatricyclo[6.2.1.0^{2,7}]undeca-2,4,6- trien-11-yl]ethyl}cyclohexyl)-2- [hydroxy(diphenyl)methyl]-1- pyrrolidinecarboxamide | R | 550.6 (2.13) |
| 67 | 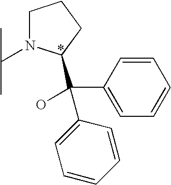 | (2S)-N-(trans-4-{2-[(1R,8S)-11- azatricyclo[6.2.1.0~2,7~]undeca- 2,4,6-trien-11-yl]ethyl}cyclohexyl)- 2-[hydroxy(diphenyl)methyl]-1- pyrrolidinecarboxamide | S | 550.6 (2.15) |

TABLE 6
Additional compounds prepared in the same manner as in Table 2.

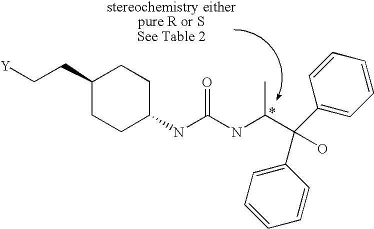

| | | | | Mass |
|---|---|---|---|---|
| | | | | Spec |
| Example | chemical name | Y | Stereo | (tR) |
| 68 | (1R,5S)-8-(2-{trans-4- [({[(1R)-2-hydroxy-1-methyl- 2,2- diphenylethyl]amino}carbonyl) amino]cyclohexyl}ethyl)-8- azabicyclo[3.2.1]oct-3-yl 3- hydroxy-2-phenylpropanoate | 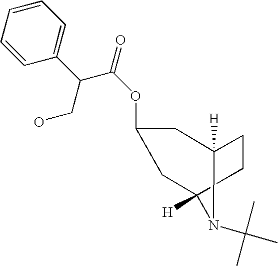 | S | 654 (1.86) |
| 69 | 1,1-dimethylethyl N-(2-{4- [({[(1S)-2-hydroxy-1-methyl- 2,2- diphenylethyl]amino}carbonyl) amino]cyclohexyl}ethyl)-N- methylglycinate | 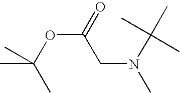 | R | 524 (1.94) |
| 70 | N-[(1S)-2-hydroxy-1-methyl- 2,2-diphenylethyl]-N′-(4-{2- [(1S)-tricyclo[3.3.1.1^{3,7}] dec-1- ylamino]ethyl}cyclohexyl) urea | 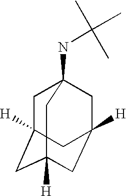 | R | 530 (2.04) |
| 71 | 1-(2-{trans-4-[({[(1R)-2- hydroxy-1-methyl-2,2- diphenylethyl]amino}carbonyl) amino]cyclohexyl}ethyl)-3- piperidinecarboxylic acid | 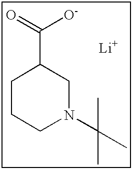 | R | 508 (1.40) |

The product of example 71 is prepared from the product of example 47 by LiOH hydrolysis.

The products of Examples 68-70 depicted in Table 5 were prepared from the product of example 6d or the S enantiomer of the product of Example 6d by the method of Example 6e with the appropriate amine. The amines were either commercially available or prepared by literature methods.

The following alternative reductive amination method was used to prepare some of the secondary amine analogs.

### Example 72

### N-(4-{2-[(diphenylmethyl)amino]ethyl}cyclohexyl)-N′-[(1R)-2-hydroxy-1-methyl-2,2-diphenylethyl]urea ( )

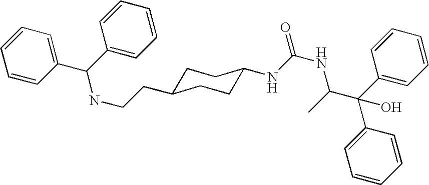

The product of Example 6d (R enantiomer), (50 mg, 0.127 mmol), and (diphenylmethyl)amine (19 uL, 0.114 mmol) were mixed in methanol (5 mL) for 60 min, then treated with sodium borohydride (5 mg, 0.127 mmol). The mixture was stirred at 23° for 24 h. The volatiles were removed in vacuo and the residue was taken up in ethyl acetate and water. Extract the water layer with ethyl acetate. The combined organic layer was dried (MgSO₄) and concentrated. The crude product was purified by SCX cartridge and reverse-phase cartridge to give 20 mg pure product. LCMS: m/z 562 (M+H), tR=1.98.

The examples 73-78 depicted in Table 6 were made by the procedure of Example 72 using the appropriate amine to afford the title compounds.

TABLE 7
Compounds made by the procedure of Example 72 and the appropriate amine.

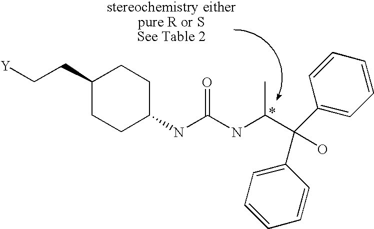

| | | | | Mass Spec |
|---|---|---|---|---|
| Example | chemical name | Y | Stereo | (tR) |
| 73 | N-[(1R)-2-hydroxy-1-methyl-2,2- diphenylethyl]-N′-[trans-4-(2-{[(1S)-1- (1- naphthalenyl)ethyl]amino}ethyl) cyclohexy]urea | 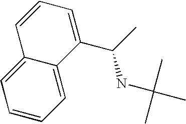 | R | 550 (1.96) |
| 74 | N-[(1R)-2-hydroxy-1-methyl-2,2- diphenylethyl]-N′-[trans-4-(2-{[(1R)-2- hydroxy-1- phenylethyl]amino}ethyl)cyclohexyl] urea | 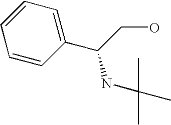 | R | 516 (1.68) |
| 75 | N-[(1R)-2-hydroxy-1-methyl-2,2- diphenylethyl]-N′-[trans-4-(2-{[(1S)-2- hydroxy-1- phenylethyl]amino}ethyl)cyclohexyl] urea | 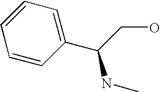 | R | 516 (1.73) |
| 76 | N-[(1R)-2-hydroxy-1-methyl-2,2- diphenylethyl]-N′-(trans-4-{2-[(1S)- 1,2,3,4-tetrahydro-1- naphthalenylamino]ethyl}cyclohexyl) urea | 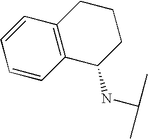 | R | 526 (1.90) |
| 77 | N-[(1R)-2-hydroxy-1-methyl-2,2- diphenylethyl]-N′-(trans-4-{2-[(1R)- 1,2,3,4-tetrahydro-1- naphthalenylamino]ethyl}cyclohexyl) urea | 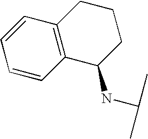 | R | 526 (1.93) |
| 78 | N-[(1R)-2-hydroxy-1-methyl-2,2- diphenylethyl]-N′-[trans-4-(2-{[(1R)-1- (2- naphthalenyl)ethyl]amino}ethyl) cyclohexyl]urea | 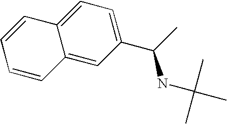 | R | 550 (1.98) |

### Example 79

### N-{4-[2-(11-azatricyclo[6.2.1.0^{2,7}]undeca-2,4,6-trien-11-yl)ethyl]cyclohexyl})-N′-[(1S)-2-cyano-1-methyl-2,2-diphenylethyl]urea

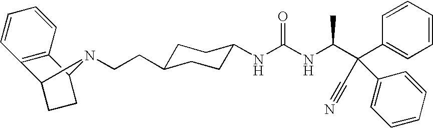

### 79a) N-[(1E)-ethylidene]-4-methylbenzenesulfinamide

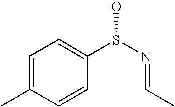

In 100 mL DCM, p-tolylsulfinamide (3.45 g, 22 mmol), acetyl aldehyde (7 mL, 111 mmol) and Ti(OEt)₄ (23 mL, 111 mmol) were stirred together 10 minutes at 0° C. and the reaction was followed by TLC. The mixture was quenched with water (100 mL) and stirred 5 minutes at 0° C. The resulted mixture was filtered through celite and the filter cake was washed with DCM. The organic phase was collected, dried (Na₂SO₄) and concentrated to afford a yellow oil which was purified by combiflash chromatography (40 g, Isco flash column) eluted with 15% to 35% EtOAc in hexane and given the title product (1.17 g, 30%)

¹H NMR (400 MHz, CDCl₃) 8.2 (q, 1H), 7.5 (d, 2H), 7.2 (d, 2H), 2.3 (s, 3H), 2.1 (d, 3H)

### 79b) N-[(1S)-2-cyano-1-methyl-2,2-diphenylethyl]-4-methylbenzenesulfinamide

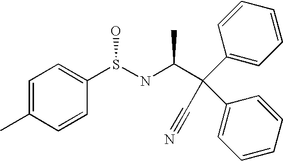

Under argon, in an oven dried flask, were placed 40 mL dry THF and 12.5 mL sodium bis(trimethylsilyl)amide (NaBSA) (0.6M solution in toluene, 7.48 mmol). The mixture was cooled to −78° C. and diphenylacetonitrile (1.446 g, 7.48 mmol) was added. After the mixture was stirred for 50 minutes, a solution of the product of example 79a, (1.1734 g, 6.48 mmol) in 20 mL dry THF was added dropwise and stirred for 5 h at −78° C.

The mixture was quenched by addition of satd aq NH₄Cl (50 mL) at −78° C. The solution was warmed to room temperature, the white precipitate filtered and rinsed with EtOAc. The filtrate was extracted with EtOAc (2×50 mL). The organic phases were washed with satd aq NaCl, dried (NaSO₄) and concentrated to give the crude product. It was purified by silica flash chromatography (20 g, SPE cartridge) washed first with hexane/EtOAc 3:1, the product was eluted with hexane/EtOAc 50:50 to give 1.6 g (66%) as a yellow oil of the S-diastereoisomer.⁽¹⁾ ⁽¹⁾J. Org. Chem, Franklin A. Davis, Ping Zhou and Bang-Chi Chen 2003, 68, 8061-8064

LCMS: m/z 375 (M+H), tR=2.25 min

### 79c) (3S)-3-amino-2,2-diphenylbutanenitrile

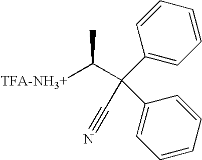

Under argon, the product of example 79b (1.6 g, 4.28 mmol) was placed in methanol (80 mL). Concentrated TFA (2 mL) was added dropwise and the mixture was stirred overnight at room temperature.

The solvent was removed, the product taken up in dichloromethane and passed through a flash chromatography (silica gel, SPE-ED 20 g) washed with 1% MeOH in CH₂Cl₂. The product was eluted in 4% MeOH and afforded 1.48 g (98%) of a white solid.

LCMS: m/z 237 (M+H)⁺, tR=1.38 min

### 79d) N-[(1S)-2-cyano-1-methyl-2,2-diphenylethyl]-N′-[4-(1,3-dioxolan-2-ylmethyl)cyclohexyl]urea

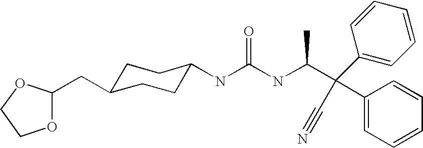

To the product of example 6b (1019 mg, 4.6 mmol) in dichloromethane/acetonitrile 1:1 (40 mL) was added diisopropylethylamine (3.2 mL, 18.4 mmol) and the solution was stirred 15 minutes. The reaction mixture was cooled to 5° C. and added to a 5° C. solution of 4-nitrophenyl chloroformate (926 mg, 4.4 mmol) in acetonitrile (20 mL) and stirred 40 min at 5° C. The resulting solution was poured into a rapidly stirred 5° C. solution of the product of example 79c (1490 mg, 4.47 mmol) in dichloromethane (40 mL). The yellow mixture was warmed to 23° and was stirred overnight.

The solution was diluted in EtOAc (200 mL), washed with saturated aq. Na₂CO₃ (3×25 mL), satd aq NaCl (25 mL), dried with Na₂SO₄ and concentrated. Purify the concentrated mixture using flash chromatography (silica gel, SPE-ED 20 g) eluted with hexane/EtOAc 5:5 afforded 1.87 g of product (94%). LCMS: m/z 448 (M+H)⁺, tR=2.18 min

### 79e) N-[(1S)-2-cyano-1-methyl-2,2-diphenylethyl]-N′-[4-(2-oxoethyl)cyclohexyl]urea

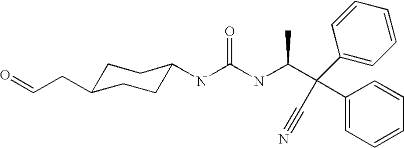

The product of Example 79d (1875 mg, 4.19 mmol) in acetone/water (9:1) (100 mL) was gently refluxed with pyridinium p-toluenesulfonate (377 mg, 1.5 mmol) over 40 h. LCMS indicated reaction was complete. The mixture was stripped to near dryness then taken up in EtOAc; washed with sat. aq. NaHCO₃, then with saturated aq NaCl; dried (Na₂SO₄), then solvent was evaporated. Purify the concentrated mixture using flash chromatography (silica gel, SPE-ED 20 g) eluted with hexane/EtOAc 1:1 and afforded 1.215 g of a white crystalline solid (72%).

LCMS: m/z 404 (M+H)⁺, tR=2.07

### 79f) N-{4-[2-(11-azatricyclo[6.2.1.0^{2,7}]undeca-2,4,6-trien-11-yl)ethyl]cyclohexyl}-N′-[(1S)-2-cyano-1-methyl-2,2-diphenylethyl]urea

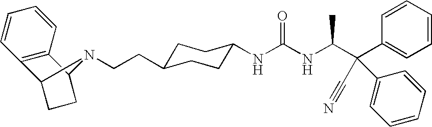

The product from Example 79e (70 mg, 0.173 mmol) in 1,2-dichloroethane (5 mL) was treated with 11-azatricyclo[6.2.1.0^{2,7}]undeca-2,4,6-triene (21.75 g, 0.15 mmol) and with rapid stirring, sodium triacetoxyborohydride (73.3 mg, 0.346 mmol) was added followed by AcOH (0.04 mL). Reaction continued to stir for an additional 17 hours. LCMS showed reaction was complete. Solvent was evaporated and the residue was taken up in EtOAc, washed with sat. aq. NaHCO₃, with sat aq. NaCl, dried (Na₂SO₄) and evaporated to a residue which was taken up in CH₂Cl₂ and passed down a ‘SCX’ [SPE-ED —5 g] column; the column was then washed with CH₂Cl₂ (60 mL), MeOH (60 mL) and finally product was eluted with 2M NH₃ in MeOH (60 mL) and afforded 88 mg of impure product. The product was passed through a Chromatotron® (1000 μg silica gel plate) eluted with MeOH in CH₂Cl₂ 1% to 5% and afforded 46 mg (58%) of a pure white solid.

LCMS: m/z 533 (M+H)⁺, tR=1.95

The products of Examples 80-85 are depicted in Table 7. These were made by the procedure of example 79 except that the amine used in example 79f was replaced by the appropriate amine to afford the title compounds in Table 7.

TABLE 8
Additional cyano analogs prepared by the method of example 79.

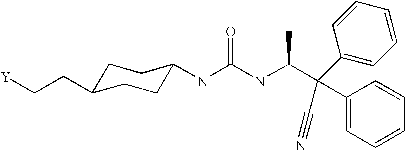

| | | | Mass spec |
|---|---|---|---|
| | | | MH⁺ |
| Example | Chemical name | Y | (tR) |
| 80 | N-(2-cyano-1-methyl-2,2-diphenylethyl)- N′-{4-[2-(3-phenyl-1- piperidinyl)ethyl]cyclohexyl}urea | 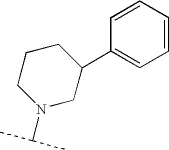 | 549 (2.05) |
| 81 | N-[(1S)-2-cyano-1-methyl-2,2- diphenylethyl]-N′-{4-[2-(2-phenyl-1- pyrrolidinyl)ethyl]cyclohexyl}urea | 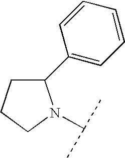 | 535 (1.98) |
| 82 | N-[(1S)-2-cyano-1-methyl-2,2- diphenylethyl]-N′-{4-[2-(3-phenyl-1- pyrrolidinyl)ethyl]cyclohexyl}urea | 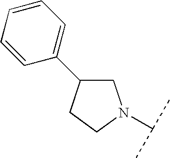 | 535 (2.03) |
| 83 | ethyl 1-(2-{trans-4-[({[(1S)-2-cyano-1- methyl-2,2- diphenylethyl]amino}carbonyl)amino] cyclohexyl}ethyl)-3-piperidinecarboxylate | 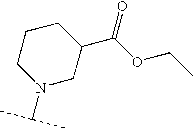 | 545 (1.75) |
| 84 | N-[(1S)-2-cyano-1-methyl-2,2- diphenylethyl]-N′-[4-(2-{[(1R)-1- phenylethyl]amino}ethyl)cyclohexyl]urea | 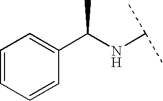 | 509 (1.90) |
| 85 | 8-(2-{4-[({[(1S)-2-cyano-1-methyl-2,2- diphenylethyl]amino}carbonyl)amino] cyclohexyl}ethyl)-8-azabicyclo[3.2.1]oct-3- yl 3-hydroxy-2-phenylpropanoate | 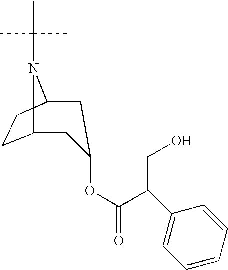 | 663 (1.95) |

### Example 86

### N-{4-[2-(11-azatricyclo[6.2.1.0^{2,7}]undeca-2,4,6-trien-11-yl)ethyl]cyclohexyl}-N′-[(1S)-2-cyano-1-methyl-2,2-diphenylethyl]urea

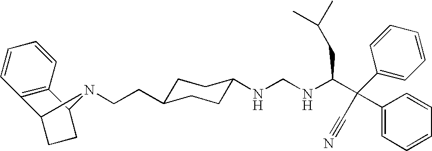

### 86a) 4-methyl-N-[(1E)-3-methylbutylidene]benzenesulfinamide

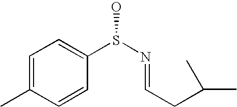

In 100 mL CH₂Cl₂, p-tolylsulfinamide (3 g, 19.3 mmol), isovaleraldehyde (10.44 mL, 96.6 mmol) and Ti(OEt)₄ (20 mL, 96.6 mmol) were stirred together 10 minutes at 0° C. and the reaction was followed by TLC. The mixture was quenched with water (100 mL) and stirred 5 minutes at 0° C. The resulted mixture was filtered through celite and the filter cake was washed with DCM. The organic phase was collected, dried and concentrated under vacuum to afford a yellow oil (3.8 g, 88%)

¹H NMR (400 MHz, CDCl₃) 8.2 (t, 1H), 7.5 (d, 2H), 7.25 (d, 2H), 2.3-2.45 (s, 3H) (m, 2H), 2(m, 1H), 0.9 (m, 6H)

### 86b) N-{(1S)-1-[cyano(diphenyl)methyl]-3-methylbutyl}4-methylbenzenesulfinamide

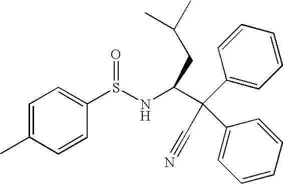

Under argon, in an oven dried flask, was placed 50 mL dry THF and 25 mL NaBSA (0.6M solution in toluene, 15 mmol). The mixture was cooled to −78° C. and diphenylacetonitrile (2.89 g, 15 mmol) was added. After the mixture was stirred for 50 minutes, a the product of example 86a (2.23 g, 10 mmol) in 20 mL dry THF was added dropwise and stirred for 5 h at −78° C.

The mixture was quenched by addition of sat. NH₄Cl (70 mL) at −78° C. The solution was warmed to room temperature, the white precipitate filtered and rinsed with EtOAc. The filtrate was extracted with EtOAc. The organic phases were washed with sat aq. NaCl, dried (NaSO₄) and concentrated to give the crude product. It was purified by Combiflash Companion (120 g ISCO silica gel column) and eluted with 0 to 30% EtOAc in hexane to give 424.7 mg (10%) as a yellow oil of the S-diastereoisomer.

LCMS: m/z 417 (M+H)⁺, tR=2.52 min

### 86c) (3S)-3-amino-5-methyl-2,2-diphenylhexanenitrile

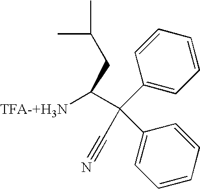

Under argon, the product of example 86c (424.7 mg, 1.02 mmol) was placed in methanol (40 mL). Concentrated TFA (1 mL) was added dropwise and the mixture was stirred overnight at room temperature.

The solvent was removed, the product taken up in dichloromethane and passed through a flash chromatography cartridge (silica gel, SPE-ED 20 g) washed with MeOH in CH₂Cl₂ 1%. The product was eluted in 4% MeOH and afforded 319.1 g (90%) of the title compound. LCMS: m/z 279 (M+H)⁺, tR=1.43 min

### 86d) N-{(1S)-1-[cyano(diphenyl)methyl]-3-methylbutyl}-N′-[4-(1,3-dioxolan-2-ylmethyl)cyclohexyl]urea

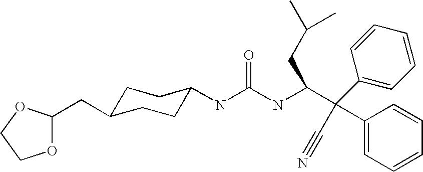

To the product of example 6b (221.72 mg, 1.15 mmol) in dichloromethane/acetonitrile 1:1 (15 mL) was added diisopropylethylamine (0.8 mL, 4.6 mmol) and the solution was stirred 15 minutes. The reaction mixture was cooled to 5° C. and added to 5° C. solution of 4-nitrophenyl chloroformate (230 mg, 1.15 mmol) in acetonitrile (7.5 mL) and stirred 40 min at 5° C. The resulting solution was poured into a rapidly stirred 5° C. solution of the product of example 86c (319.1 mg, 1.15 mmol) in dichloromethane (15mL). The yellow mixture was warmed to RT and was stirred overnight.

The solution was diluted in EtOAc (50 mL), washed with satd aq Na₂CO₃ (3×10 mL), sat aq NaCl (10 mL), dried with Na₂SO₄ and concentrated to afford 571 mg of the crude product (99%). LCMS: f/z 490 (M+H)⁺, tR=1.95 min

### 86e) N-{(1S)-1-[cyano(diphenyl)methyl]-3-methylbutyl}-N′-[4-(2-oxoethyl)cyclohexyl]urea

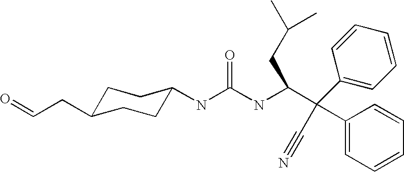

The product of Example 86d (489.66 mg, 1.16 mmol) in acetone/water (9:1) 30 mL was gently refluxed with pyridinium p-toluenesulfonate (97.7 mg, 0.38 mmol) over 5 days. The mixture was stripped to near dryness then taken up in EtOAc, washed with sat. aq. NaHCO₃, sat aq. NaCl, dried (Na₂SO₄), then solvent was evaporated. Purify the concentrated mixture using chromatotron (4000 μg silica gel) eluted with hexane/EtOAc 1:1 and afforded 218 mg of a white crystalline solid. (42%). LCMS: m/z 446 (M+H)⁺, tR=2.38 min.

### 86f) N-{4-[2-(11-azatricyclo[6.2.1.0^{2,7}]undeca-2,4,6-trien-11-yl)ethyl]cyclohexyl}-N′-[(1S)-2-cyano-1-methyl-2,2-diphenylethyl]urea

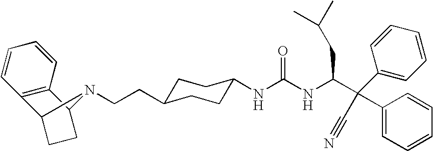

The product from Example 86e (70 mg, 0.157 mmol) in 1,2-dichloroethane (4 mL) was treated with 11-azatricyclo[6.2.1.0^{2,7}]undeca-2,4,6-triene (21 g, 0.145 mmol) and with rapid stirring, sodium triacetoxyborohydride (73.3 mg, 0.346 mmol) was added followed by AcOH (0.04 mL). Reaction continued to stir for an additional 20 hours. LCMS showed the reaction was complete. Reaction mixture was diluted in CH₂Cl₂, washed with sat. aq. NaHCO₃, with sat aq. NaCl and passed down a ‘SCX’ [SPE-ED —1 g] column; the column was then washed with CH₂Cl₂ (20 mL), MeOH (20 mL) and finally product was eluted with 2M NH₃ in MeOH (40 mL) and afforded 75.9 mg of impure product. The product was passed through a chromatotron (1000 g silica gel plate) eluted with MeOH in CH₂Cl₂ 1% to 5% and afforded 25.5 mg (30%) of a pure white solid. LCMS: m/z 575 (M+H)⁺, Tr=2.25 min.

The products of Examples 87-88 are depicted in Table 8. These were made by the procedure of example 86 except that the amine used in example 86f was replaced by the appropriate amine to afford the title compounds in Table 8.

TABLE 9
Additional cyano analogs prepared by the method of example 86.

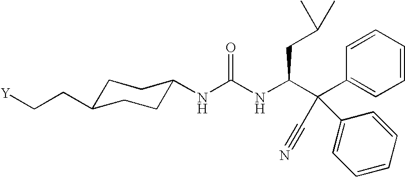

| | | | Mass spec |
|---|---|---|---|
| Example | Chemical name | Y | (tR) |
| 87 | 8-[2-(4-{[({(1S)-1- [cyano(diphenyl)methyl]-3- methylbutyl}amino)carbonyl]amino} cyclohexyl)ethyl]-8- azabicyclo[3.2.1]oct-3-yl 3- hydroxy-2-phenylpropanoate | 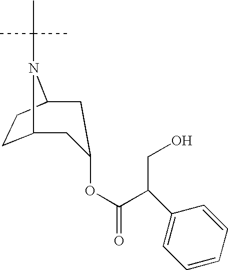 | 706 (2.25) |
| 88 | ethyl 1-[2-(trans-4-{[({(1S)-1- [cyano(diphenyl)methyl]-3- methylbutyl}amino)carbonyl]amino} cyclohexyl)ethyl]-3- piperidinecarboxylate | 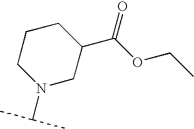 | 587 (2.25) |

TABLE 10
Examples 89-96 were made from the indicated 1,1-diaryl-1-hydroxy amino alcohol
intermediate from Table 4 by the methods of Example 6d and 6e.

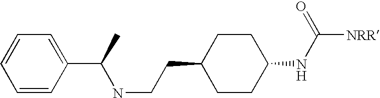

| | | | | Mass |
|---|---|---|---|---|
| | NRR′ | | stereoHR | spec |
| example | (Intermediate) | chemical name | at R | (tR) |
| 89 | 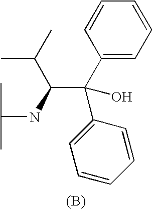 | N-{(1S)-1-[hydroxy(diphenyl)methyl]-2- methylpropyl}-N′-[trans-4-(2-{[(1R)-1- phenylethyl]amino}ethyl)cyclohexyl]urea | S | 528.6 (1.91) |
| 90 | 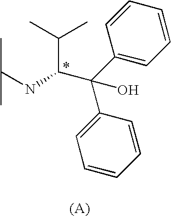 | N-{(1R)-1-[hydroxy(diphenyl)methyl]-2- methylpropyl}-N′-[trans-4-(2-{[(1R)-1- phenylethyl]amino}ethyl)cyclohexyl]urea | R | 528.6 (1.93) |
| 91 | 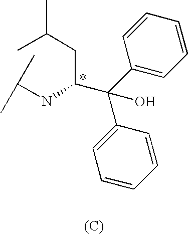 | N-{(1R)-1-[hydroxy(diphenyl)methyl]-3- methylbutyl}-N′-[trans-4-(2-{[(1R)-1- phenylethyl]amino}ethyl)cyclohexyl]urea | R | 524.8 (2.01) |
| 92 | 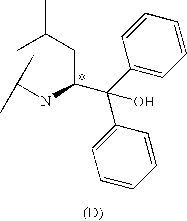 | N-{(1S)-1-[hydroxy(diphenyl)methyl]-3- methylbutyl}-N′-[trans-4-(2-{[(1R)-1- phenylethyl]amino}ethyl)cyclohexyl]urea | S | 542.6 (2.00) |
| 93 | 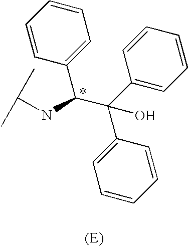 | N-[(1S)-2-hydroxy-1,2,2-triphenylethyl]- N′-[trans-4-(2-{[(1R)-1- phenylethyl]amino}ethyl)cyclohexyl]urea | S | 562.4 (1.92) |
| 94 | 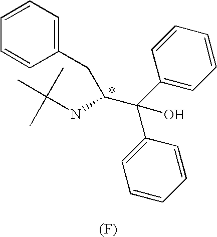 | N-[(1R)-2-hydroxy-2,2-diphenyl-1- (phenylmethyl)ethyl]-N′-[trans-4-(2- {[(1R)-1- phenylethyl]amino}ethyl)cyclohexyl]urea | R | 576.6 (1.99) |
| 95 | 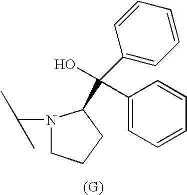 | (2R)-2-[hydroxy(diphenyl)methyl]-N- [trans-4-(2-{[(1R)-1- phenylethyl]amino}ethyl)cyclohexyl]-1- pyrrolidinecarboxamide | R | 526.2 (1.99) |
| 96 | 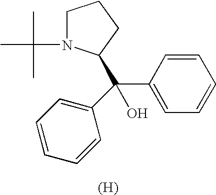 | (2S)-2-[hydroxy(diphenyl)methyl]-N- [trans-4-(2-{[(1R)-1- phenylethyl]amino}ethyl)cyclohexyl]-1- pyrrolidinecarboxamide | S | 526.8 (2.07) |

TABLE 11
Examples 97-102 were made from the indicated 1,1-diaryl-1-hydroxy amino alcohol
intermediate from Table 4 by the methods of Example 6d and 6e except that the amine used in the
reductive amination step corresponding to example 6e, to make the title (example) compounds,
was (1R,5S)-8-azabicyclo[3.2.1]oct-3-yl 3-hydroxy-2-phenylpropanoate.

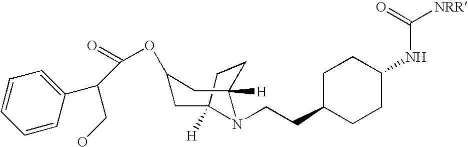

| | | | | Mass |
|---|---|---|---|---|
| | NRR′ | | Stereochem | spec |
| example | (Intermediate) | chemical name | at R | (tR) |
| 97 | 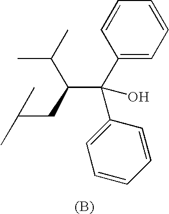 | (1R,5S)-8-[2-(trans-4-{[({(1S)-1- [hydroxy(diphenyl)methyl]-2- methylpropyl}amino)carbonyl]amino} cyclohexyl)ethyl]-8- azabicyclo[3.2.1]oct-3-yl 3-hydroxy-2- phenylpropanoate | S | 682.0 (1.97) |
| 98 | 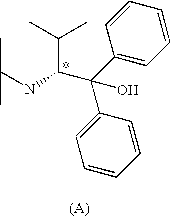 | (1R,5S)-8-[2-(trans-4-{[({(1R)-1- [hydroxy(diphenyl)methyl]-2- methylpropyl}amino)carbonyl]amino} cyclohexyl)ethyl]-8- azabicyclo[3.2.1]oct-3-yl 3-hydroxy-2- phenylpropanoate | R | 682.4 (1.96) |
| 99 | 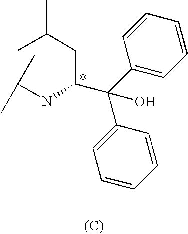 | (1R,5S)-8-[2-(trans-4-{[({(1R)-1- [hydroxy(diphenyl)methyl]-3- methylbutyl}amino)carbonyl]amino} cyclohexyl)ethyl]-8- azabicyclo[3.2.1]oct-3-yl 3-hydroxy-2- phenylpropanaote | R | 696.6 (2.09) |
| 100 | 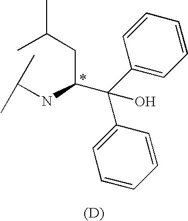 | (1R,5S)-8-[2-(trans-4-{[({(1S)-1- [hydroxy(diphenyl)methyl]-3- methylbutyl}amino)carbonyl]amino} cyclohexyl)ethyl]-8- azabicyclo[3.2.1]oct-3-yl 3-hydroxy-2- phenylpropanoate | S | 696.6 (2.00) |
| 101 | 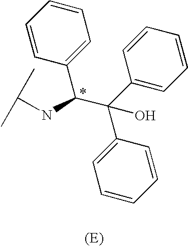 | (1R,5S)-8-(2-{trans-4-[({[(1S)-2- hydroxy-1,2,2- triphenylethyl]amino}carbonyl)amino] cyclohexyl}ethyl)-8- azabicyclo[3.2.1]oct-3-yl 3-hydroxy-2- phenylpropanoate | S | 717 (2.04) |
| 102 | 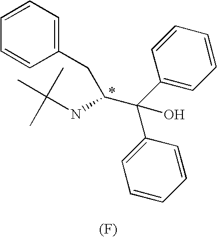 | (1R,5S)-8-(2-{trans-4-[({[(1R)-2- hydroxy-2,2-diphenyl-1- (phenylmethyl)ethyl]amino}carbonyl) amino]cyclohexyl}ethyl)-8- azabicyclo[3.2.1]oct-3-yl 3-hydroxy-2- phenylpropanoate | R | 730.6 (2.05) |

TABLE 12
Additional Compounds made by the procedure of Example 72 and the
appropriate amine.

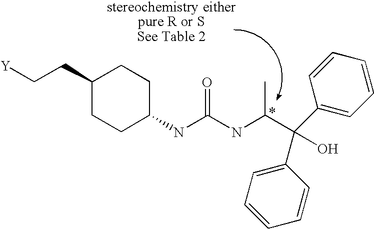

| | | | | Mass Spec |
|---|---|---|---|---|
| Example | chemical name | Y | Stereo | (tR) |
| 103 | N-[trans-4-(2-{[(1S,2R)-2-hydroxy- 2,3-dihydro-1H-inden-1- yl]amino}ethyl)cyclohexyl]-N′- [(1R)-2-hydroxy-1-methyl-2,2- diphenylethyl]urea | 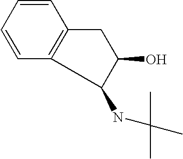 | R | 527 (1.87) |
| 104 GSK655561 | N-[(1R)-2-hydroxy-1-methyl-2,2- diphenylethyl]-N′-{trans-4-[2-({3- methyl 2-[(1Z)-1-propen-1-yl]-1H- inden-1- yl)amino)ethyl]cyclohexyl}urea | 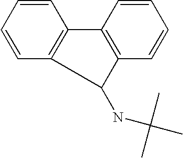 | R | 560 (2.01) |
| 105 | N-[trans-4-(2-{[(1R,2S)-2-hydroxy- 2,3-dihydro-1H-inden-1- yl]amino}ethyl)cyclohexyl]-N′- [(1R)-2-hydroxy-1-methyl-2,2- diphenylethyl]urea | 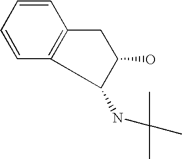 | R | 528 (1.70) |
| 106 | N-[(1R)-2-hydroxy-1-methyl-2,2- diphenylethyl]-N′-(trans-4-{2-[(1- methyl-1- phenylethyl)amino]ethyl}cyclohexyl) urea | 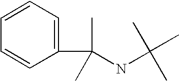 | R | 514 (1.77) |
| 107 | N-[(1R)-2-hydroxy-1-methyl-2,2- diphenylethyl]-N′-[trans-4-(2- {[(1S)-2-(4-methylphenyl)-1- phenylethyl]amino}ethyl)cyclohexyl] urea | 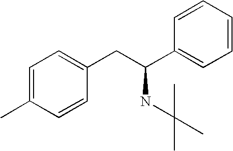 | R | 590 (1.99) |

### Example 108

### N-{4-[2-(11-azatricyclo[6.2.1.0^{2,7}]undeca-2,4,6-trien-11-yl)ethyl]cyclohexyl}-N′-[(1S)-2-cyano-1-methyl-2,2-diphenylethyl]urea ( )

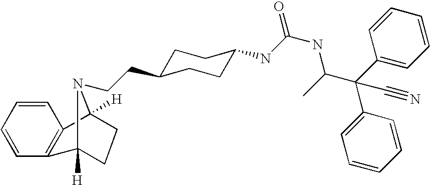

AlCl₃ (201 mg, 1.5 mol) was added to N-{4-[2-(11-azatricyclo[6.2.1.0²′]undeca-2,4,6-trien-11-yl)ethyl]cyclohexyl}-N′-[(1S)-2-hydroxy-1-methyl-2,2-diphenylethyl]urea (the S enantiomer of product of example 43) (159 mg, 0.3 mmol) in DCE (2 mL) at RT. After stirring 30 minutes, TMSCN (0.16 mL, 1.5 mmol) was added. After 1 hr, add aq. NaHCO₃ and ethyl acetate, separate the layers and dry the organic layer (MgSO₄), then concentrate. Purify by reverse-phase cartridge to give 15 mg product, LCMS: m/z 533 (M+H)⁺, tR=2.04.

### Biological Examples

The inhibitory effects of compounds at the mAChRs of the present invention are determined by the following in vitro and in vivo functional assays:

### Analysis of Inhibition of Receptor Activation by Calcium Mobilization:

Stimulation of mAChRs expressed on CHO cells were analyzed by monitoring receptor-activated calcium mobilization as previously described (4). CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels (5). The emitted fluorescence from all 96 wells is measured simultaneously using a cooled CCD camera. Data points are collected every second. This data was then plotting and analyzed using GraphPad PRISM software.

### Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine (2). Mice were pretreated with 50 μl of compound (0.003-10 μg/mouse) in 50 μl of vehicle (10% DMSO) intranasally, i.v., i.p. or p.o, and were then placed in the plethysmography chamber. Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis; gastrointestinal-tract disorders such as irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakisuria, neurogenic bladder, nocturnal enuresis, psychosomatic bladder, incontinence associated with bladder spasms or chronic cystitis, urinary urgency or pollakiuria, and motion sickness.

Methods of administering the present compounds will be readily apparent to the skilled artisan.

Dry powder compositions for topical delivery to the lung by inhalation may, for example, be presented in capsules and cartridges of for example gelatine, or blisters of for example laminated aluminium foil, for use in an inhaler or insulator. Formulations generally contain a powder mix for inhalation of the compound of the invention and a suitable powder base (carrier substance) such as lactose or starch. Use of lactose is preferred. Each capsule or cartridge may generally contain between 20 μg-10 mg of the compound of formula (I) optionally in combination with another therapeutically active ingredient. Alternatively, the compound of the invention may be presented without excipients.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognised as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

A preferable means for applying the formulation of the present invention to the nasal passages is by use of a pre-compression pump. Most preferably, the pre-compression pump will be a VP7 model manufactured by Valois S A. Such a pump is beneficial as it will ensure that the formulation is not released until a sufficient force has been applied, otherwise smaller doses may be applied. Another advantage of the pre-compression pump is that atomisation of the spray is ensured as it will not release the formulation until the threshold pressure for effectively atomising the spray has been achieved. Typically, the VP7 model may be used with a bottle capable of holding 10-50 ml of a formulation. Each spray will typically deliver 50-100 μg of such a formulation, therefore, the VP7 model is capable of providing at least 100 metered doses.

### Examples of Nasal Formulations

### Example 1

### Nasal Formulation Containing Active

A formulation for intranasal delivery was prepared with ingredients as follows:

| | to 100% | |
|---|---|---|
| Active | 0.1% | w/w |
| Polysorbate 80 | 0.025% | w/w |
| Avicel RC591 | 1.5% | w/w |
| Dextrose | 5.0% | w/w |
| BKC | 0.015% | w/w |
| EDTA | 0.015% | w/w |

| | |
|---|---|
| water | to 100% |

in a total amount suitable for 120 actuations and the formulation was filled into a bottle fitted with a metering valve adapted to dispense 50 or 100 μl per actuation.
The device was fitted into a nasal actuator (Valois).

### Example 2

### Nasal Formulation Containing Active

A formulation for intranasal delivery was prepared with ingredients as follows:

| | | |
|---|---|---|
| Active | 0.005% | w/w |
| Tyloxapol | 2% | w/w |
| dextrose | 5% | w/w |
| BKC | 0.015% | w/w |
| EDTA | 0.015% | w/w |

| | |
|---|---|
| water | to 100% |

in a total amount suitable for 120 actuations and the formulation was filled into a bottle (plastic or glass) fitted with a metering valve adapted to dispense 50 or 100 μl per actuation

The device was fitted into a nasal actuator (Valois, e.g. VP3, VP7 or VP7D)

### Example 3

### Nasal Formulation Containing Active

A formulation for intranasal delivery was prepared with ingredients as follows:

| | | |
|---|---|---|
| active | 0.05% | w/w |
| Triton X-100 | 5% | w/w |
| Dextrose | 4% | w/w |
| BKC | 0.015% | w/w |
| EDTA | 0.015% | w/w |

| | |
|---|---|
| water | to 100% |

in a total amount suitable for 120 actuations and the formulation was filled into a bottle fitted with a metering valve adapted to dispense 50 or 100 μl per actuation.

### Example 4

### Nasal Formulation Containing Active

A formulation for intranasal delivery was prepared with ingredients as follows:

| | | |
|---|---|---|
| active | 0.05% | w/w |
| Tyloxapol | 5% | w/w |
| dextrose | 5% | w/w |
| BKC | 0.015% | w/w |
| EDTA | 0.015% | w/w |

| | |
|---|---|
| water | to 100% |

in a total amount suitable for 120 actuations and the formulation was filled into a bottle fitted with a metering valve adapted to dispense 50 or 100 μl per actuation
The device was fitted into a nasal actuator (Valois).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

What is claimed is:

1. A compound according to Formula (I) herein below: 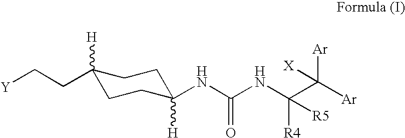
wherein:
cyclohexyl stereochemistry is cis or trans;
Y is an amine, or quaternary amine salt;
R⁴ and R⁵ are independently selected from the group consisting of a substituent selected from C₁₋₆ alkyl, aryl and arylC₁₋₆alkyl, C₁₋₆ alkylaryl, heteroaryl, heteroalkyl, all optionally substituted, and H;
X is OH or CN;
amine is selected from the group consisting of: 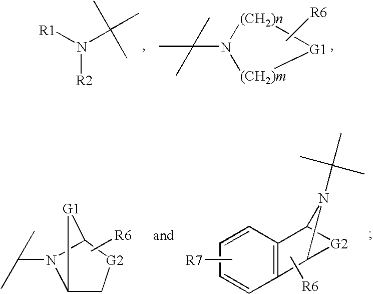
R¹ and R² are independently selected from a group consisting of H, alkyl, cycloalkyl, aryl, alkylaryl, alkylalkenyl, arylalkyl and arylalkenyl, all optionally substituted;
m is an integer from 0 to 6;
n is an integer from 0 to 5;
G1 and G2 are independently selected from, bond, (CH2)p, O, NR1, —CR1=CR1-, S(O)p, CO and CONR1-;
p is an integer from 0 to 2;
R6 and R7 are independently selected from ═R1, F, Cl, Br, CN, OR1, OCOR2, NR1R2 and NCOR2;
Ar is independently selected from the group consisting of aryl and heteroaryl, all optionally substituted;
Quaternary ammonium salt is selected from the group consisting of: 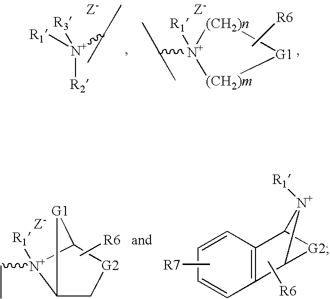
Z- is an anionic counterion;
R₁′, R₂′ and R₃′, are independently selected from a group consisting of H, alkyl, cycloalkyl, aryl, alkylaryl, alkylalkenyl, arylalkyl, and arylalkenyl, all optionally substituted,
and M, N, G1. G2 P, R6 and R7 are as defined herein above.
and pharmaceutically acceptable salts thereof.

2. A compound according to claim 1 consisting of the group selected from the group consisting of:
N-(trans-4-{2-[(1R,8S)-11-azatricyclo[6.2.1.0˜2,7˜]undeca-2,4,6-trien-11-yl]ethyl}cyclohexyl)-N′-[(1S)-2-hydroxy-1-methyl-2,2-diphenylethyl]urea;
N-(trans-4-{2-[bis(phenylmethyl)amino]ethyl}cyclohexyl)-N′-[(1S)-2-hydroxy-1-methyl-2,2-diphenylethyl]urea;
N-(trans-4-{2-[bis(phenylmethyl)amino]ethyl}cyclohexyl)-N′-[(1R)-2-hydroxy-1-methyl-2,2-diphenylethyl]urea;
N-{trans-4-[2-(dicyclohexylamino)ethyl]cyclohexyl}-N′-[(1R)-2-hydroxy-1-methyl-2,2-diphenylethyl]urea;
N-{trans-4-[2-(dicyclohexylamino)ethyl]cyclohexyl}-N′-[(1S)-2-hydroxy-1-methyl-2,2-diphenylethyl]urea;
N-(trans-4-{2-[(1R,8S)-11 azatricyclo[6.2.1.0˜2,7˜]undeca-2,4,6-trien-11-yl]ethyl}cyclohexyl)-N′-[(1R)-2-hydroxy-1-methyl-2,2-diphenylethyl]urea;
N-(trans-4-{2-[(1R,5S)-3-azabicyclo[3.2.1]oct-3-yl]ethyl}cyclohexyl)-N′-[(1S)-2-hydroxy-1-methyl-2,2-diphenylethyl]urea;
N-(trans-4-{2-[(1R,4S)-2-azabicyclo[2.2.1]hept-2-yl]ethyl}cyclohexyl)-N′-[(1R)-2-hydroxy-1-methyl-2,2-diphenylethyl]urea;
N-(trans-4-{2-[(1R,5S)-8-azabicyclo[3.2.1]oct-8-yl]ethyl}cyclohexyl)-N′-[(1R)-2-hydroxy-1-methyl-2,2-diphenylethyl]urea;
N-{trans-4-[2-(1,3-dihydro-2H-isoindol-2-yl)ethyl]cyclohexyl}N′-[(1R)-2-hydroxy-1-methyl-2,2-diphenylethyl]urea;
N-{trans-4-[2-(2,3-dihydro-1H-indol-1-yl)ethyl]cyclohexyl}-N′-[(1R)-2-hydroxy-1-methyl-2,2-diphenylethyl]urea;
N-(trans-4-{2-[(1s,4s)-7-azabicyclo[2.2.1]hept-7-yl]ethyl}cyclohexyl)-N′-[(1R)-2-hydroxy-1-methyl-2,2-diphenylethyl]urea;
N-[(1R)-2-hydroxy-1-methyl-2,2-diphenylethyl]-N′-(trans-4-{2-[7-(2-methylpropanoyl)-1,2,4,5-tetrahydro-3H-3-benzazepin-3-yl]ethyl}cyclohexyl)urea;
N-(trans-4-{2-[(1s,5s)-3-azabicyclo[3.2.2]non-3-yl]ethyl}cyclohexyl)-N′-[(1R)-2-hydroxy-1-methyl-2,2-diphenylethyl]urea;
N-(trans-4-{2-[(1R,5S)-3-azabicyclo[3.2.1]oct-3-yl]ethyl}cyclohexyl)-N′-[(1R)-2-hydroxy-1-methyl-2,2-diphenylethyl]urea;
N-(trans-4-{2-[(1R,5S)-3-hydroxy-8-azabicyclo[3.2.1]oct-8-yl]ethyl}cyclohexyl)-N′-[(1R)-2-hydroxy-1-methyl-2,2-diphenylethyl]urea;
Ethyl N-(2-{trans-4-[({[(1R)-2-hydroxy-1-methyl-2,2-diphenylethyl]amino}carbonyl)amino]cyclohexyl}ethyl)-N-methylglycinate;
N-{trans-4-[2-(3-azabicyclo[3.2.2]non-3-yl)ethyl]cyclohexyl}-N′-[(1S)-2-hydroxy-1-methyl-2,2-diphenylethyl]urea;
N-(trans-4-{2-[(1R,4S)-2-azabicyclo[2.2.1]hept-2-yl]ethyl}cyclohexyl)-N′-[(1S)-2-hydroxy-1-methyl-2,2-diphenylethyl]urea;
N-(trans-4-{2-[(1s,4s)-7-azabicyclo[2.2.1]hept-7-yl]ethyl}cyclohexyl)-N′-[(1S)-2-hydroxy-1-methyl-2,2-diphenylethyl]urea;
N-(trans-4-{2-[(1R,5S)-8-azabicyclo[3.2.1]oct-8-yl]ethyl}cyclohexyl)-N′-[(1S)-2-hydroxy-1-methyl-2,2-diphenylethyl]urea;
(1R,5S)-8-(2-{trans-4-[({[(1S)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
(1R,2S,4R,5S)-9-(2-{trans-4-[({[(1S)-2-hydroxy-1-methyl-2,2-diphenylethyl]amino}carbonyl)amino]cyclohexyl}ethyl)-3-oxa-9-azatricyclo[3.3.1.0˜2,4˜]non-7-yl 3-hydroxy-2-phenylpropanoate;
N-{trans-4-[2-(3,4-dihydro-1(2H)-quinolinyl)ethyl]cyclohexyl}-N′-[(1S)-2-hydroxy-1-methyl-2,2-diphenylethyl]urea;
N-{trans-4-[2-(3,4-dihydro-2(1H)-isoquinolinyl)ethyl]cyclohexyl}-N′-[(1S)-2-hydroxy-1-methyl-2,2-diphenylethyl]urea;
N-(trans-4-{2-[(1R,5S)-3-hydroxy-8-azabicyclo[3.2.1]oct-8-yl]ethyl}cyclohexyl)-N′-[(1S)-2-hydroxy-1-methyl-2,2-diphenylethyl]urea;
N-[(1S)-2-hydroxy-1-methyl-2,2-diphenylethyl]-N′-{trans-4-[2-(2-phenyl-1-pyrrolidinyl)ethyl]cyclohexyl}urea;
N-[(1S)-2-hydroxy-1-methyl-2,2-diphenylethyl]-N′-{trans-4-[2-(4-phenyl-1-piperidinyl)ethyl]cyclohexyl}urea;
N-(trans-4-{2-[(1R,8S)-4-azatricyclo[4.3.1.1˜3,8˜]undec-4-yl]ethyl}cyclohexyl)-N′-[(1R)-2-hydroxy-1-methyl-2,2-diphenylethyl]urea;
1,1-dimethylethyl N-(2-{trans-4-[({[(1R)-2-hydroxy-1-methyl-2,2-diphenylethyl]amino}carbonyl)amino]cyclohexyl}ethyl)-N-methylglycinate;
N-[(1R)-2-hydroxy-1-methyl-2,2-diphenylethyl]-N′-{trans-4-[2-(2-phenyl-1-piperidinyl)ethyl]cyclohexyl}urea;
N-[(1R)-2-hydroxy-1-methyl-2,2-diphenylethyl]-N′-(trans-4-{2-[methyl(phenylmethyl)amino]ethyl}cyclohexyl)urea;
N-[(1R)-2-hydroxy-1-methyl-2,2-diphenylethyl]-N′-{trans-4-[2-(2-phenyl-1-pyrrolidinyl)ethyl]cyclohexyl}urea;
N-(2-{trans-4-[({[(1R)-2-hydroxy-1-methyl-2,2-diphenylethyl]amino}carbonyl)amino]cyclohexyl}ethyl)-N-methylglycine;
N-[(1R)-2-hydroxy-1-methyl-2,2-diphenylethyl]-N′-{trans-4-[2-(3-phenyl-1-pyrrolidinyl)ethyl]cyclohexyl}urea;
N-[(1R)-2-hydroxy-1-methyl-2,2-diphenylethyl]-N′-{trans-4-[2-(3-phenyl-1-piperidinyl)ethyl]cyclohexyl}urea;
9-(2-{trans-4-[({[(1R)-2-hydroxy-1-methyl-2,2-diphenylethyl]amino}carbonyl)amino]cyclohexyl}ethyl)-3-oxa-9-azatricyclo[3.3.1.0˜2,4˜]non-7-yl 3-hydroxy-2-phenylpropanoate;
N-[(1S)-2-hydroxy-1-methyl-2,2-diphenylethyl]-N′-(trans-4-{2-[(1R,5S)-3-hydroxy-3-phenyl-8-azabicyclo[3.2.1]oct-8-yl]ethyl}cyclohexyl)urea;
Ethyl N-(2-{trans-4-[({[(1S)-2-hydroxy-1-methyl-2,2-diphenylethyl]amino}carbonyl)amino]cyclohexyl}ethyl)-N-methylglycinate;
N-[(1S)-2-hydroxy-1-methyl-2,2-diphenylethyl]-N′-(trans-4-{2-[7-(2-methylpropanoyl)-1,2,4,5-tetrahydro-3H-3-benzazepin-3-yl]ethyl}cyclohexyl)urea;
N-[(1S)-2-hydroxy-1-methyl-2,2-diphenylethyl]-N′-{trans-4-[2-(3-phenyl-1-piperidinyl)ethyl]cyclohexyl}urea;
1-[(1S)-2-hydroxy-1-methyl-2,2-diphenylethyl]-3-{trans-4-[2-(3-phenyl-1-piperidinyl)ethyl]cyclohexyl}urea;
N-[(1S)-2-hydroxy-1-methyl-2,2-diphenylethyl]-N′-{trans-4-[2-(3-phenyl-1-pyrrolidinyl)ethyl]cyclohexyl}urea;
N-[(1S)-2-hydroxy-1-methyl-2,2-diphenylethyl]-N′-{trans-4-[2-(2-phenyl-1-piperidinyl)ethyl]cyclohexyl}urea;
(1R,8S)-11-(2{trans-4-[({[(1R)-2-hydroxy-1-methyl-2,2-diphenylethyl]amino}carbonyl)amino]cyclohexyl}ethyl)-11-methyl-11-azoniatricyclo[6.2.1.0˜2,7˜]undeca-2,4,6-triene iodide;
(1R,8S)-11-(2-{trans-4-[({[(1S)-2-hydroxy-1-methyl-2,2-diphenylethyl]amino}carbonyl)amino]cyclohexyl}ethyl)-11-methyl-11-azoniatricyclo[6.2.1.0˜2,7˜]undeca-2,4,6-triene iodide;
1-(2-{trans-4-[({[(1R)-2-hydroxy-1-methyl-2,2-diphenylethyl]amino}carbonyl)amino]cyclohexyl}ethyl)-1-methyl-3-phenylpiperidinium iodide;
2-(2-{trans-4-[({[(1S)-2-hydroxy-1-methyl-2,2-diphenylethyl]amino}carbonyl)amino]cyclohexyl}ethyl)-2-methyl-2-azoniatricyclo[3.3.1.1˜3,7˜]decane iodide;
N-[(1R)-2-hydroxy-1-methyl-2,2-diphenylethyl]-N′-(trans-4-{2-[(1R,8S)-4-(phenylacetyl)-11-azatricyclo[6.2.1.0˜2,7˜]undeca-2,4,6-trien-11-yl]ethyl}cyclohexyl)urea;
3-(2-{trans-4-[({[(1R)-2-hydroxy-1-methyl-2,2-diphenylethyl]amino}carbonyl)amino]cyclohexyl}ethyl)-3-methyl-7-(2-methylpropanoyl)-2,3,4,5-tetrahydro-1H-3-benzazepinium iodide;
Ethyl 1-(2-{trans-4-[({[(1S)-2-hydroxy-1-methyl-2,2-diphenylethyl]amino}carbonyl)amino]cyclohexyl}ethyl)-3-piperidinecarboxylate;
lithium 1-(2-{trans-4-[({[(1S)-2-hydroxy-1-methyl-2,2-diphenylethyl]amino}carbonyl)amino]cyclohexyl}ethyl)-3-piperidinecarboxylate;
N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-2-methylpropyl}urea;
N-(trans-4--{2-[(1R,8S)-11-azatricyclo[6.2.1.0˜2,7˜]undeca-2,4,6-trien-11-yl]ethyl}cyclohexyl)-N′-{(1R)-1-[hydroxy(diphenyl)methyl]-2-methylpropyl}urea;
N-(trans-4-{2-[(1R,8S)-11-azatricyclo[6.2.1.0˜2,7˜]undeca-2,4,6-trien-11-yl]ethyl}cyclohexyl)-N′-{(1R)-1-[hydroxy(diphenyl)methyl]-3-methylbutyl}urea;
N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-3-methylbutyl}urea;
N-(trans-4-{2-[(1R,8S)-11-azatricyclo[6.2.1.0˜2,7˜]undeca-2,4,6-trien-11-yl]ethyl}cyclohexyl)-N′-[(1S)-2-hydroxy-1,2,2-triphenylethyl]urea;
N-(trans-4-{2-[(1R,8S)-11-azatricyclo[6.2.1.0˜2,7˜]undeca-2,4,6-trien-11-yl]ethyl}cyclohexyl)-N′-[(1R)-2-hydroxy-2,2-diphenyl-1-phenylmethyl)ethyl]urea;
(2R)—N-(trans-4-{2-[(1R,8S)-11-azatricyclo[6.2.1.0˜2,7˜]undeca-2,4,6-trien-11-yl]ethyl}cyclohexyl)-2-[hydroxy(diphenyl)methyl]-1-pyrrolidinecarboxamide;
(2S)—N-(trans-4-{2-[(1R,8S)-11-azatricyclo[6.2.1.0˜2,7˜]undeca-2,4,6-trien-1-yl]ethyl}cyclohexyl)-2-[hydroxy(diphenyl)methyl]-1-pyrrolidinecarboxamide; 1,1-dimethylethyl N-(2-{4-[({[(1S)-2-hydroxy-1-methyl-2,2-diphenylethyl]amino}carbonyl)amino]cyclohexyl}ethyl)-N-methylglycinate;
1-[(1S)-2-hydroxy-1-methyl-2,2-diphenylethyl]-3-(4-{2-[(1S,3R,5R,7S)-tricyclo[3.3.1.1^{3,7}]dec-1-ylamino]ethyl}cyclohexyl)urea;
1-[(1R)-2-hydroxy-1-methyl-2,2-diphenylethyl]-3-(trans-4-{2-[(phenylmethyl)amino]ethyl}cyclohexyl)urea;
1-[(1R)-2-hydroxy-1-methyl-2,2-diphenylethyl]-3-[trans-4-(2-{([(1R)-1-phenylethyl]amino}ethyl)cyclohexyl]urea;
(1R,5S)-8-(2-{4-[({[(1S)-2-hydroxy-1-methyl-2,2-diphenylethyl]amino}carbonyl)amino]cyclohexyl}ethyl)-3-[(3-hydroxy-2-phenylpropanoyl)oxy]-8-methyl-8-azoniabicyclo[3.2.1]octane iodide;
1-(trans-4-{2-[(1R)-2,3-dihydro-1H-inden-1-ylamino]ethyl}cyclohexyl)-3-[(1R)-2-hydroxy-1-methyl-2,2-diphenylethyl]urea trifluoroacetate (salt);
1-(trans-4-{2-[(1S)-2,3-dihydro-1H-inden-1-ylamino]ethyl}cyclohexyl)-3-[(1R)-2-hydroxy-1-methyl-2,2-diphenylethyl]urea;
(1R,5S)-8-(2-{trans-4-[({[(1R)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
1-(trans-4-{2-[(diphenylmethyl)amino]ethyl}cyclohexyl)-3-[(1R)-2-hydroxy-1-methyl-2,2-diphenylethyl]urea;
1-[(1R)-2-hydroxy-1-methyl-2,2-diphenylethyl]-3-[trans-4-(2-{[(1S)-1-(1-naphthalenyl)ethyl]amino}ethyl)cyclohexyl]urea;
1-[trans-4-(2-{[(1S,2R)-2-hydroxy-2,3-dihydro-1H-inden-1-yl]amino}ethyl)cyclohexyl]-3-[(1R)-2-hydroxy-1-methyl-2,2-diphenylethyl]urea;
1-{(1S)-1-[hydroxy(diphenyl)methyl]-2-methylpropyl}-3-[trans-4-(2-{[(1R)-1-phenylethyl]amino}ethyl)cyclohexyl]urea;
1-{1R)-1-[hydroxy(diphenyl)methyl]-2-methylpropyl}-3-[trans-4-(2-{[(1R)-1-phenylethyl]amino}ethyl)cyclohexyl]urea;
1-{(1R)-1-[hydroxy(diphenyl)methyl]-3-methylbutyl}-3-[trans-4-(2-{([(1R)-1-phenylethyl]amino}ethyl)cyclohexyl]urea;
1-{(1S)-1-[hydroxy(diphenyl)methyl]-3-methylbutyl}-3-[trans-4-(2-{([(1R)-1-phenylethyl]amino}ethyl)cyclohexyl]urea;
1-[(1S)-2-hydroxy-1,2,2-triphenylethyl]-3-[trans-4-(2-{[(1R)-1-phenylethyl]amino}ethyl)cyclohexyl]urea;
1-[(1R)-2-hydroxy-2,2-diphenyl-1-(phenylmethyl)ethyl]-3-[trans-4-(2-{[(1R)-1-phenylethyl]amino}ethyl)cyclohexyl]urea;
(2R)-2-[hydroxy(diphenyl)methyl]-N-[trans-4-(2-{[(1R)-1-phenylethyl]amino}ethyl)cyclohexyl]-1-pyrrolidinecarboxamide;
(2S)-2-[hydroxy(diphenyl)methyl]-N-[trans-4-(2-{[(1R)-1-phenylethyl]amino}ethyl)cyclohexyl]-1-pyrrolidinecarboxamide;
Ethyl 1-(2-{trans-4-[({[(1R)-2-hydroxy-1-methyl-2,2-diphenylethyl]amino}carbonyl)amino]cyclohexyl}ethyl)-3-piperidinecarboxylate;
1-(2-(trans-4-[({[(1R)-2-hydroxy-1-methyl-2,2-diphenylethyl]amino}carbonyl)amino]cyclohexyl}ethyl)-3-piperidinecarboxylic acid trifluoroacetate (salt);
1-{trans-4-[2-(9H-fluoren-9-ylamino)ethyl]cyclohexyl}-3-[(1R)-2-hydroxy-1-methyl-2,2-diphenylethyl]urea;
1-[trans-4-(2-{[(1R,2S)-2-hydroxy-2,3-dihydro-1H-inden-1-yl]amino}ethyl)cyclohexyl]-3-[(1R)-2-hydroxy-1-methyl-2,2-diphenylethyl]urea;
1-[(1R)-2-hydroxy-1-methyl-2,2-diphenylethyl]-3-[trans-4-(2-{([(1R)-2-hydroxy-1-phenylethyl]amino}ethyl)cyclohexyl]urea;
1-[(1R)-2-hydroxy-1-methyl-2,2-diphenylethyl]-3-[trans-4-(2-{[(1S)-2-hydroxy-1-phenylethyl]amino}ethyl)cyclohexyl]urea;
(1R,5S)-8-[2-(trans-4-{[({(1S)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[{(1R)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[({(1R)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[({(1S)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
(1R,5S)-8-(2-{trans-4-[({[(1S)-2-hydroxy-1,2,2-triphenylethyl]amino}carbonyl)amino]cyclohexyl}ethyl)-8-azabicyclo[3.2.1]oct-3-yl 3-hydroxy-2-phenylpropanoate;
(1R,5S)-8-(2-{trans-4-[({[(1R)-2-hydroxy-2,2-diphenyl-1-(phenylmethyl)ethyl]amino}carbonyl)amino]cyclohexyl}ethyl)-8-azabicyclo[3.2.1]oct-3-yl 3-hydroxy-2-phenylpropanoate;
1-[(1R)-2-hydroxy-1-methyl-2,2-diphenylethyl]-3-(trans-4-{2-[(1S)-1,2,3,4-tetrahydro-1-naphthalenylamino]ethyl}cyclohexyl)urea;
1-[(1R)-2-hydroxy-1-methyl-2,2-diphenylethyl]-3-(trans-4-{2-[(1R)-1,2,3,4-tetrahydro-1-naphthalenylamino]ethyl}cyclohexyl)urea;
1-[(1R)-2-hydroxy-1-methyl-2,2-diphenylethyl]-3-[trans-4-(2-{[(1R)-1-(2-naphthalenyl)ethyl]amino}ethyl)cyclohexyl]urea;
1-[(1R)-2-hydroxy-1-methyl-2,2-diphenylethyl]-3-[trans-4-(2-{[(1S)-2-(4-methylphenyl)-1-phenylethyl]amino}ethyl)cyclohexyl]urea;
1-[(1R)-2-hydroxy-1-methyl-2,2-diphenylethyl]-3-(trans-4-{2-[(1-methyl-1-phenylethyl)amino]ethyl}cyclohexyl)urea;
1-(trans-4-{2-[(1R,8S)-11-azatricyclo[6.2.1.0^{2,7}]undeca-2,4,6-trien-11-yl]ethyl}cyclohexyl)-3-(2-cyano-1-methyl-2,2-diphenylethyl)urea;
1-(trans-4-{2-[(1R,8S)-11-azatricyclo[6.2.1.0^{2,7}]undeca-2,4,6-trien-11-yl]ethyl}cyclohexyl)-3-[(1S)-2-cyano-1-methyl-2,2-diphenylethyl]urea;
1-[(1S)-2-cyano-1-methyl-2,2-diphenylethyl]-3-{4-[2-(3-phenyl-1-piperidinyl)ethyl]cyclohexyl}urea;
1-[(1S)-2-cyano-1-methyl-2,2-diphenylethyl]-3-{4-[2-(2-phenyl-1-pyrrolidinyl)ethyl]cyclohexyl}urea;
1-[(1S)-2-cyano-1-methyl-2,2-diphenylethyl]-3-{trans-4-[2-(3-phenyl-1-pyrrolidinyl)ethyl]cyclohexyl}urea;
Ethyl 1-(2-{trans-4-[({[(1S)-2-cyano-1-methyl-2,2-diphenylethyl]amino}carbonyl)amino]cyclohexyl}ethyl)-3-piperidinecarboxylate;
1-[(1S)-2-cyano-1-methyl-2,2-diphenylethyl]-3-[trans-4-(2-{[(1R)-1-phenylethyl]amino}ethyl)cyclohexyl]urea;
(1R,5R)-8-(2-{trans-4-[({[(1S)-2-cyano-1-methyl-2,2-diphenylethyl]amino}carbonyl)amino]cyclohexyl}ethyl)-1,5-dimethyl-8-azabicyclo[3.2.1]oct-3-yl 3-hydroxy-2-phenylpropanoate;
1-(trans-4-{2-[(1R,8S)-1-azatricyclo[6.2.1.0^{2,7}] undeca-2,4,6-trien-11-yl]ethyl}cyclohexyl)-3-{(1S)-1-[cyano(diphenyl)methyl]-3-methylbutyl}urea;
(1S,5S)-8-[2-(trans-4-{[({(1S)-1-[cyano(diphenyl)methyl]-3-methylbutyl}amino)carbonyl]amino}cyclohexyl)ethyl]-8-azabicyclo[3.2.1]oct-3-yl 3-hydroxy-2-phenylpropanoate; and
Ethyl 1-[2-(trans-4-{[({(1S)-1-[cyano(diphenyl)methyl]-3-methylbutyl}amino)carbonyl]amino}cyclohexyl)ethyl]-3-piperidinecarboxylate′;
and pharmaceutically acceptable salts thereof.

3. A compound according to claim 2 consisting of the group selected from:
(1R,5S)-8-(2{trans-4-[({[(1S)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
(1R,5S)-8-[2-(trans-4-{[({(1R)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
N-(trans-4-{2-[(1R,8S)-11-azatricyclo[6.2.1.0˜2,7˜]undeca-2,4,6-trien-11-yl]ethyl}cyclohexyl)-N′-{(1R)-1-[hydroxy(diphenyl)methyl]-3-methylbutyl}urea;
ethyl 1-(2-{trans-4-[({[(1S)-2-hydroxy-1-methyl-2,2-diphenylethyl]amino}carbonyl)amino]cyclohexyl}ethyl)-3-piperidinecarboxylate;
N-[(1R)-2-hydroxy-1-methyl-2,2-diphenylethyl]-N′-(trans-4-{2-[7-(2-methylpropanoyl)-1,2,4,5-tetrahydro-3H-3-benzazepin-3-yl]ethyl}cyclohexyl)urea;
Ethyl 1-[2-(trans-4-{[({(1S)-1-[cyano(diphenyl)methyl]-3-methylbutyl}amino)carbonyl]amino}cyclohexyl)ethyl]-3-piperidinecarboxylate′;
N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-3-methylbutyl}urea;
N-(trans-4-{2-[(1R,8S)-11-azatricyclo[6.2.1.0˜2,7˜]undeca-2,4,6-trien-11-yl]ethyl}cyclohexyl)-N′-[(1R)-2-hydroxy-2,2-diphenyl-1-(phenylmethyl)ethyl]urea;
1-(trans-4-{2-[(1S)-2,3-dihydro-1H-inden-1-ylamino]ethyl}cyclohexyl)-3-[(1R)-2-hydroxy-1-methyl-2,2-diphenylethyl]urea;
(1R,5S)-8-(2-{trans-4-[({[(1R)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
(1R,5S)-8-(2-{trans-4-[({[(1R)-2-hydroxy-1-methyl-2,2-diphenylethyl]amino}carbonyl)amino]cyclohexyl}ethyl)-8-azabicyclo[3.2.1]oct-3-yl 3-hydroxy-2-phenylpropanoate;
(1S,5S)-8-[2-(trans-4-{[({(1S)-1-[cyano(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
N-(trans-4-{2-[(1R,8S)-11-azatricyclo[6.2.1.0˜2,7˜]undeca-2,4,6-trien-11-yl]ethyl}cyclohexyl)-N′-[(1S)-2-hydroxy-1,2,2-tripheriylethyl]urea;
N-(trans-4-{2-[(1R,8S)-11-azatricyclo[6.2.1.0˜2,7˜]undeca-2,4,6-trien-11-yl]ethyl}cyclohexyl)-N′-{(1R)-1-[hydroxy(diphenyl)methyl]-2-methylpropyl}urea;
N-[(1S)-2-hydroxy-1-methyl-2,2-diphenylethyl]-N′-(trans-4-{2-[7-(2-methylpropanoyl)-1,2,4,5-tetrahydro-3H-3-benzazepin-3-yl]ethyl}cyclohexyl)urea;
Ethyl 1-(2-{trans-4-[({[(1S)-2-cyano-1-methyl-2,2-diphenylethyl]amino}carbonyl)amino]cyclohexyl}ethyl)-3-piperidinecarboxylate;
1-[(1R)-2-hydroxy-1-methyl-2,2-diphenylethyl]-3-(trans-4-{2-[(1-methyl-1-phenylethyl)amino]ethyl}cyclohexyl)urea;
(1R,5S)-8-(2-{4-[({[(1S)-2-hydroxy-1-methyl-2,2-diphenylethyl]amino}carbonyl)amino]cyclohexyl}ethyl)-3-[(3-hydroxy-2-phenylpropanoyl)oxy]-8-methyl-8-azoniabicyclo[3.2.1]octane iodide;
N-(trans-4-{2-[(1R,8S)-11-azatricyclo[6.2.1.0˜2,7˜]undeca-2,4,6-trien-11-yl]ethyl}cyclohexyl)-N′-{(1S)-1-[hydroxy(diphenyl)methyl]-2-methylpropyl}urea;
(2R)—N-(trans-4-{2-[(1R,8S)-11-azatricyclo[6.2.1.0˜2,7˜]undeca-2,4,6-trien-11-yl]ethyl}cyclohexyl)-2-[hydroxy(diphenyl)methyl]-1-pyrrolidinecarboxamide;
N-[(1R)-2-hydroxy-1-methyl-2,2-diphenylethyl]-N′-{trans-4-[2-(3-phenyl-1-piperidinyl)ethyl]cyclohexyl}urea;
1{(1R)-1-[hydroxy(diphenyl)methyl]-3-methylbutyl}-3-[trans-4-(2-{[(1R)-1-phenylethyl]amino}ethyl)cyclohexyl]urea; (1R,5R)-8-(2-{trans-4-[({[(1S)-2-cyano-{-methyl-2,2-diphenylethyl]amino}carbonyl)amino]cyclohexyl}ethyl)-1,5-dimethyl-8-azabicyclo[3.2.1]oct-3-yl 3-hydroxy-2-phenylpropanoate;
N-[(1R)-2-hydroxy-1-methyl-2,2-diphenylethyl]-N′-{trans-4-[2-(3-phenyl-1-pyrrolidinyl)ethyl]cyclohexyl}urea;
9-(2-{trans-4-[({[(1R)-2-hydroxy-1-methyl-2,2-diphenylethyl]amino}carbonyl)amino]cyclohexyl}ethyl)-3-oxa-9-azatricyclo[3.3.1.0˜2,4˜]non-7-yl 3-hydroxy-2-phenylpropanoate;
1-[(1R)-2-hydroxy-2,2-diphenyl-1-(phenylmethyl)ethyl]-3-[trans-4-(2-{[(1R)-1-phenylethyl]amino}ethyl)cyclohexyl]urea;
1-(trans-4-{2-[(1R,8S)-11-azatricyclo[6.2.1.0^{2,7}]undeca-2,4,6-trien-11-yl]ethyl}cyclohexyl)-3-{(1S)-1-[cyano(diphenyl)methyl]-3-methylbutyl}urea;
1-(trans-4-{2-[(1R,8S)-11-azatricyclo[6.2.1.0^{2,7}]undeca-2,4,6-trien-11-yl]ethyl}cyclohexyl)-3-(2-cyano-1-methyl-2,2-diphenylethyl)urea;
1-(trans-4-{2-[(1R,8S)-11-azatricyclo[6.2.1.0^{2,7}]undeca-2,4,6-trien-11-yl]ethyl}cyclohexyl)-3-(2-cyano-1-methyl-2,2-diphenylethyl)urea;
1-{(1S)-1-[hydroxy(diphenyl)methyl]-3-methylbutyl}-3-[trans-4-{(2-([(1R)-1-phenylethyl]amino}ethyl)cyclohexyl]urea;
′N-(trans-4-{2-[(1R,8S)-1-azatricyclo[6.2.1.0˜2,7˜]undeca-2,4,6-trien-11-yl]ethyl}cyclohexyl)-N′-[(1S)-2-hydroxy-1-methyl-2,2-diphenylethyl]urea;
1-[(1S)-2-hydroxy-1,2,2-triphenylethyl]-3-[trans-4-(2-{[(1R)-1-phenylethyl]amino}ethyl)cyclohexyl]urea;
(1R,2S,4R,5S)-9-(2-{trans-4-[({[(1S)-2-hydroxy-1-methyl-2,2-diphenylethyl]amino}carbonyl)amino]cyclohexyl}ethyl)-3-oxa-9-azatricyclo[3.3.1.0˜2,4˜]non-7-yl 3-hydroxy-2-phenylpropanoate;
N-(trans-4-{2-[(1R,8S)-4-azatricyclo[4.3.1.1˜3,8˜]undec-4-yl]ethyl}cyclohexyl)-N′-[(R)-2-hydroxy-1-methyl-2,2-diphenylethyl]urea;
1-[(1R)-2-hydroxy-1-methyl-2,2-diphenylethyl]-3-[trans-4-(2-{[(1R)-1-phenylethyl]amino}ethyl)cyclohexyl]urea;
1-{(1S)-1-[hydroxy(diphenyl)methyl]-2-methylpropyl}-3-[trans-4-(2-{([(1R)-1-phenylethyl]amino}ethyl)cyclohexyl]urea;
1-{(1R)-1-[hydroxy(diphenyl)methyl]-2-methylpropyl}-3-[trans-4-(2-{([(1R)-1-phenylethyl]amino}ethyl)cyclohexyl]urea;
N-[(1S)-2-hydroxy-1-methyl-2,2-diphenylethyl]-N′-{trans-4-[2-(3-phenyl-1-pyrrolidinyl)ethyl]cyclohexyl}urea;
1-[(1S)-2-cyano-1-methyl-2,2-diphenylethyl]-3-{4-[2-(2-phenyl-1-pyrrolidinyl)ethyl]cyclohexyl}urea;
1-(trans-4-{2-[(1R,8S)-11-azatricyclo[6.2.1.0^{2,7}]undeca-2,4,6-trien-11-yl]ethyl}cyclohexyl)-3-[(1S)-2-cyano-1-methyl-2,2-diphenylethyl]urea;
(1R,8S)-11-(2-{trans-4-[({[(1R)-2-hydroxy-1-methyl-2,2-diphenylethyl]amino}carbonyl)amino]cyclohexyl}ethyl)-11-methyl-11-azoniatricyclo[6.2.1.0˜2,7˜]undeca-2,4,6-triene iodide;
N-(trans-4-{2-[(1R,5S)-8-azabicyclo[3.2.1]oct-8-yl]ethyl}cyclohexyl)-N′-[(1S)-2-hydroxy-1-methyl-2,2-diphenylethyl]urea;
N-(trans-4-{2-[(1R,5S)-8-azabicyclo[3.2.1]oct-8-yl]ethyl}cyclohexyl)-N′-[(1R)-2-hydroxy-1-methyl-2,2-diphenylethyl]urea;
N-{trans-4-[2-(3-azabicyclo[3.2.2]non-3-yl)ethyl]cyclohexyl}-N′-[(1S)-2-hydroxy-1-methyl-2,2-diphenylethyl]urea;
1-[(1S)-2-cyano-1-methyl-2,2-diphenylethyl]-3-{trans-4-[2-(3-phenyl-1-pyrrolidinyl)ethyl]cyclohexyl}urea;
(1R,8S)-11-(2-{trans-4-[({[(1S)-2-hydroxy-1-methyl-2,2-diphenylethyl]amino}carbonyl)amino]cyclohexyl}ethyl)-11-methyl-11-azoniatricyclo[6.2.1.0˜2,7˜]undeca-2,4,6-triene iodide;
1-[(1S)-2-hydroxy-1-methyl-2,2-diphenylethyl]-3-{trans-4-[2-(3-phenyl-1-piperidinyl)ethyl]cyclohexyl}urea;
2-(2-{trans-4-[({[(1S)-2-hydroxy-1-methyl-2,2-diphenylethyl]amino}carbonyl)amino]cyclohexyl}ethyl)-2-methyl-2-azoniatricyclo[3.3.1.1˜3,7˜]decane iodide;
1-[(1R)-2-hydroxy-1-methyl-2,2-diphenylethyl]-3-[trans-4-(2-{[(1S)-2-hydroxy-1-phenylethyl]amino}ethyl)cyclohexyl]urea;
3-(2-{trans-4-[({[(1R)-2-hydroxy-1-methyl-2,2-diphenylethyl]amino}carbonyl)amino]cyclohexyl}ethyl)-3-methyl-7-(2-methylpropanoyl)-2,3,4,5-tetrahydro-1H-3-benzazepinium iodide;
N-trans-4-(4-{2-[(1s,4s)-7-azabicyclo[2.2.1]hept-7-yl]ethyl}cyclohexyl)-N′-[(1S)-2-hydroxy-1-methyl-2,2-diphenylethyl]urea;
N-trans-4-{2-[(1s,4s)-7-azabicyclo[2.2.1]hept-7-yl]ethyl}cyclohexyl)-N′-[(1R)-2-hydroxy-1-methyl-2,2-diphenylethyl]urea;
1-[(1S)-2-hydroxy-1-methyl-2,2-diphenylethyl]-3-(4-{2-[(1S,3R,5R,7S)-tricyclo[3.3.1.1^{3,7}]dec-1-ylamino]ethyl}cyclohexyl)urea;
1,1-dimethylethyl N-(2-{4-[({[(1S)-2-hydroxy-1-methyl-2,2-diphenylethyl]amino}carbonyl)amino]cyclohexyl}ethyl)-N-methylglycinate;
N-[(1R)-2-hydroxy-1-methyl-2,2-diphenylethyl]-N′-{trans-4-[2-(2-phenyl-1-piperidinyl)ethyl]cyclohexyl}urea;
(1R,5S)-8-[2-(trans-4-{[({(1S)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[({(1S)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
(1R,5S)-8-(2-{trans-4-[({[(1S)-2-hydroxy-1,2,2-triphenylethyl]amino}carbonyl)amino]cyclohexyl}ethyl)-8-azabicyclo[3.2.1]oct-3-yl 3-hydroxy-2-phenylpropanoate;
(1R,5S)-8-(2-{trans-4-[({[(1R)-2-hydroxy-2,2-diphenyl-1-(phenylmethyl)ethyl]amino}carbonyl)amino]cyclohexyl}ethyl)-8-azabicyclo[3.2.1]oct-3-yl 3-hydroxy-2-phenylpropanoate; and
(1R,5S)-8-[2-(trans-4-{[({(1R)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
and pharmaceutically acceptable salts thereof.

4. A compound according to claim 3 selected from the group consisting of:
(1R,5S)-8-[2-(trans-4-{[({(18)-1-[hydroxy(diphenyl)methyl]-3-methylbutyl}amino)carbonyl]amino}cyclohexyl)ethyl]-8-azabicyclo[3.2.1]oct-3-yl 3-hydroxy-2-phenylpropanoate;
(1R,5S)-8-[2-(trans-4-{[({(1S)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
(1R,5S)-8-(2-{trans-4-[({[(1S)-2-hydroxy-1,2,2-triphenylethyl]amino}carbonyl)amino]cyclohexyl}ethyl)-8-azabicyclo[3.2.1]oct-3-yl 3-hydroxy-2-phenylpropanoate;
(1R,5S)-8-(2-{trans-4-[({[(1R)-2-hydroxy-2,2-diphenyl-1-(phenylmethyl)ethyl]amino}carbonyl)amino]cyclohexyl}ethyl)-8-azabicyclo[3.2.1]oct-3-yl 3-hydroxy-2-phenylpropanoate; and
(1R,5S)-8-[2-(trans-4-{[({(1R)-1-[hydroxy(diphenyl)methyl]-2-methylpropyl}amino)carbonyl]amino}cyclohexyl)ethyl]-8-azabicyclo[3.2.1]oct-3-yl 3-hydroxy-2-phenylpropanoate;
and pharmaceutically acceptable salts thereof.

5. A pharmaceutical composition for the treatment of muscarinic acetylcholine receptor mediated diseases comprising a compound according to claim 1 and a pharmaceutically acceptable carrier thereof.

6. A method of inhibiting the binding of acetylcholine to its receptors in a mammal in need thereof comprising administering a safe and effective amount of a compound according to claim 1.

7. A method of treating a muscarinic acetylcholine receptor mediated disease, wherein acetylcholine binds to said receptor, comprising administering a safe and effective amount of a compound according to claim 1.

8. A method according to claim 7 wherein the disease is selected from the group consisting of chronic obstructive lung disease, chronic bronchitis, asthma, chronic respiratory obstruction, pulmonary fibrosis, pulmonary emphysema and allergic rhinitis.

9. A method according to claim 8 wherein administration is via inhalation via the mouth or nose.

10. A method according to claim 9 wherein administration is via a medicament dispenser selected from a reservoir dry powder inhaler, a multi-dose dry powder inhaler or a metered dose inhaler.

11. A method according to claim 10 wherein the compound is administered to a human and has a duration of action of 12 hours or more for a 1 mg dose.

12. A method according to claim 11 wherein the compound has a duration of action of 24 hours or more.

13. A method according to claim 12 wherein the compound has a duration of action of 36 hours or more.
